SEQUENCE LISTING (5 sequences)

SEQ ID NO: 1 - protein, 110 aa, Homo sapiens
PVLVKATETLTLTCTVSGFSLSNAGMGVSWIRQPPGKALEWLAHIFSNDEKSYSTSLERRLTISKDTSKSQVVLTMTNMDPVDTGTYYCARTRQPYNQNMDVWGQGTLVT

SEQ ID NO: 2 - protein, 106 aa, Homo sapiens
QTVVTQEPSFSVSPGGTVTLTCALSSGSVSPSYYPSWSQQTPGQAPRTLIYSTNIRSSGVPDRFSGSVLGNKAALTITGAQAEDESDYYCMLYVGSAIWVFGGGTK

SEQ ID NO: 3 - protein, 333 aa, Homo sapiens
GGGTGACCAGGGTTCCTTGGCCCCAGACGTCCATATTCTGGTTATACGGCTGACGGGTCCGTGCACAATAATATGTGCCTGTGTCCACGGGGTCCATGTTGGTCATGGTAAGGACCACCTGGCTTTTGGAGGTGTCCTTGGAGATGGTGAGTCTTCTCTCCAGAGATGTGCTGTAGGATTTTTCGTCATTCGAAAAAATGTGTGCAAGCCACTCCAGGGCCTTCCCTGGGGGCTGACGGATCCAGCTCACACCCATTCCAGCATTGCTGAGTGAGAACCCAGAGACGGTGCAGGTCAGCGTGAGGGTCTCTGTGGCTTTCACCAGCACAGGAC

SEQ ID NO: 4 - protein, 318 aa, Homo sapiens
CAGACTGTGGTGACCCAGGAGCCATCGTTCTCAGTGTCCCCTGGAGGGACAGTCACACTCACTTGTGCCTTGAGCTCTGGCTCAGTCTCTCCTAGTTATTACCCCAGCTGGTCCCAGCAGACTCCAGGCCAGGCTCCACGCACACTCATCTACAGCACAAATATTCGCTCTTCTGGGGTCCCTGATCGCTTCTCTGGCTCCGTCCTTGGGAACAAAGCTGCCCTCACCATCACGGGGGCCCAGGCTGAAGATGAGTCTGATTATTATTGTATGTTGTATGTGGGTAGTGCCATTTGGGTGTTCGGCGGAGGGACCAAG

SEQ ID NO: 5 - protein, 272 aa, Homo sapiens
MAAKVFESIGKFGLALAVAGGVVNSALYNVDAGHRAVIFDRFRGVQDIVVGEGTHFLIPWVQKPIIFDCRSRPRNVPVITGSKDLQNVNITLRILFRPVASQLPRIFTSIGEDYDERVLPSITTEILKSVVARFDAGELITQRELVSRQVSDDLTERAATFGLILDDVSLTHLTFGKEFTEAVEAKQVAQQEAERARFVVEKAEQQKKAAIISAEGDSKAAELIANSLATAGDGLIELRKLEAAEDIAYQLSRSRNITYLPAGQSVLLQLPQ

(19) United States
(12) Patent Application Publication
(10) Pub. No.: US 2009/0258020 A1
(43) Pub. Date: Oct. 15, 2009

(54) ANTIBODY DESIGNATED BARB3, BARB3 RELATED ANTIBODIES, AND METHODS OF MAKING AND USING SAME

(75) Inventors: Heinz Peter Vollmers, Wurzburg (DE); Stephanie Ute Brandlein, Zellingen (DE); Frank Hensel, Wurzburg (DE)

Correspondence Address: PILLSBURY WINTHROP SHAW PITTMAN LLP, ATTENTION: DOCKETING DEPARTMENT, P.O BOX 10500, McLean, VA, 22102, US

(73) Assignee: PATRYS LIMITED, Melbourne (AU)

(21) Appl. No.: 12/349,390

(22) Filed: Jan. 6, 2009

Related U.S. Application Data
(60) Provisional application No. 61/019,477, filed on Jan. 7, 2008.
(60) Provisional application No. 61/043,961, filed on Apr. 10, 2008.

(51) Int. Cl. A61K 39/395; C07K 16/30; C12N 5/10; C07K 14/435; A61P 35/00

(52) U.S. Cl. 424/138.1; 530/387.7; 530/387.3; 435/325; 530/350

(57) ABSTRACT

The invention provides antibodies, functional fragments, modified and variant forms, nucleic acid and other compositions. Antibodies, functional fragments, modified and variant forms, nucleic acid and other compositions are useful in treatment and diagnostic methods. One treatment method includes inhibiting growth or proliferation of proliferating cells, such as hyperproliferative cells or inducing regression of hyperproliferative cells, such as cells of a cellular hyperproliferative disorder.

### RELATED APPLICATIONS

This application claims priority to application Ser. No. 61/019,477, filed Jan. 7, 2008, and application Ser. No. 61/043,961, filed Apr. 10, 2008, each of which are incorporated by reference herein in their entirety.

### FIELD OF THE INVENTION

The invention relates to an antibody, known as BARB3. The antibody denoted BARB3 is an IgG and binds to different types of neoplasia, cancer, tumor and metastasis. BARB3 inhibits growth of various types of cancer cells and stimulates or induces apoptosis of various types of cancer cells.

### INTRODUCTION

There is a need for methods of treating undesirable or aberrant cell proliferation, such as cellular hyperproliferative disorders, including neoplasias, tumors, cancers and metastasis. The invention addresses this need and provides related benefits.

### SUMMARY

The invention provides isolated and purified antibodies and functional fragments that compete for binding to a cell or to an antigen that intact BARB3 antibody (as represented by DSMZ Deposit No. DSM ACC2859, or heavy and light chain sequences set forth as SEQ ID NOs:1 and 2) binds. In one embodiment, an antibody or functional fragment competes with BARB3 antibody, as represented by DSMZ Deposit No. DSM ACC2859, or heavy and light chain sequences set forth as SEQ ID NOs:1 and 2, for binding of to an adenocarcinoma cell or a squamous cell carcinoma. In particular aspects, an antibody or functional fragment competes with BARB3 antibody, as represented by DSMZ Deposit No. DSM ACC2859, or heavy and light chain sequences set forth as SEQ ID NOs:1 and 2, for binding to one or more of a stomach adenocarcinoma cell, a lung adenocarcinoma cell, a pancreas adenocarcinoma cell, a colon adenocarcinoma cell, a breast adenocarcinoma cell, or an esophagus squamous cell carcinoma. In another embodiment, an antibody or functional fragment competes with BARB3 antibody, as represented by DSMZ Deposit No. DSM ACC2859, or heavy and light chain sequences set forth as SEQ ID NOs:1 and 2, for binding to a human adenocarcinoma, human squamous cell carcinoma, human carcinoid carcinoma, human invasive ductal carcinoma, or human germ cell carcinoma of any of stomach, lung, colon, pancreas, esophagus, prostate, breast or testis. In an additional embodiment, an antibody or functional fragment competes with BARB3 antibody, as represented by DSMZ Deposit No. DSM ACC2859, or heavy and light chain sequences set forth as SEQ ID NOs:1 and 2, for binding to a pancreas cancer cell line BXPC-3 (ATCC Deposit No. CRL-1687) or a stomach cancer cell line 23132/87 (DSMZ Deposit No. ACC 201). In a further embodiment, an antibody or functional fragment thereof inhibits or reduces proliferation, or stimulates or induces apoptosis, of one or more of a stomach adenocarcinoma cell, a lung adenocarcinoma cell, a pancreas adenocarcinoma cell, a colon adenocarcinoma cell, a breast adenocarcinoma cell, or an esophagus squamous cell carcinoma, a human adenocarcinoma, human squamous cell carcinoma, human carcinoid carcinoma, human invasive ductal carcinoma, or a human germ cell carcinoma in any of stomach, lung, colon, pancreas, esophagus, prostate, breast or testis, or a pancreas cancer cell line BXPC-3 (ATCC Deposit No. CRL-1687) or a stomach cancer cell line 23132/87 (DSMZ Deposit No. ACC 201).

The invention also provides isolated and purified antibodies and functional fragments thereof that bind to cells or to an antigen that BARB3 antibody (as represented by DSMZ Deposit No. DSM ACC2859, or heavy and light chain sequences set forth as SEQ ID NOs:1 and 2) binds. In one embodiment, an antibody or functional fragment binds to an adenocarcinoma cell or a squamous cell carcinoma to which BARB3 antibody, as represented by DSMZ Deposit No. DSM ACC2859, or heavy and light chain sequences set forth as SEQ ID NOs:1 and 2, binds. In particular aspects, an antibody or functional fragment binds to one or more of a stomach adenocarcinoma cell, a lung adenocarcinoma cell, a pancreas adenocarcinoma cell, a colon adenocarcinoma cell, a breast adenocarcinoma cell, an esophagus squamous cell carcinoma, to which BARB3 antibody, as represented by DSMZ Deposit No. DSM ACC2859, or heavy and light chain sequences set forth as SEQ ID NOs:1 and 2, binds. In another embodiment, an antibody or functional fragment binds to a human adenocarcinoma, human squamous cell carcinoma, human carcinoid carcinoma, human invasive ductal carcinoma, or human germ cell carcinoma of any of stomach, lung, colon, pancreas, esophagus, prostate, breast or testis to which BARB3 antibody, as represented by DSMZ Deposit No. DSM ACC2859, or heavy and light chain sequences set forth as SEQ ID NOs:1 and 2, binds. In an additional embodiment, an antibody or functional fragment binds to a pancreas cancer cell line BXPC-3 (ATCC Deposit No. CRL-1687) or a stomach cancer cell line 23132/87 (DSMZ Deposit No. ACC 201) that BARB3 antibody, as represented by DSMZ Deposit No. DSM ACC2859, or heavy and light chain sequences set forth as SEQ ID NOs:1 and 2, binds.

The invention further provides isolated and purified antibodies and functional fragments that include a heavy or light chain variable region sequence with about 60% or more identity to a heavy or light chain sequence variable regions as represented by DSMZ Deposit No. DSM ACC2859, or as set forth in SEQ ID NOs:1 or 2. In one embodiment, an antibody or subsequence thereof includes a sequence at least 60% or more (e.g., 65%, 70%, 75%, 80%, 85%, 90%, 95%, etc.) identical to a heavy chain variable region sequence set forth as SEQ ID NO:1, or a sequence at least 60% or more (e.g., 65%, 70%, 75%, 80%, 85%, 90%, 95%, etc.) identical to a light chain variable region sequence set forth as SEQ ID NO:2. In another embodiment, an antibody or subsequence includes a sequence at least 60% or more (e.g., 65%, 70%, 75%, 80%, 85%, 90%, 95%, etc.) identical to a heavy chain variable region sequence set forth as SEQ ID NO:1, and a sequence at least 60% or more (e.g., 65%, 70%, 75%, 80%, 85%, 90%, 95%, etc.) identical to a light chain variable region sequence set forth as SEQ ID NO:2. In a further embodiment, an antibody or subsequence includes a sequence at least 80-85%, 85-90%, 90-95%, or 95-100% identical to one or more CDRs in heavy chain variable region sequence set forth as SEQ ID NO:1, or a sequence at least 80-85%, 85-90%, 90-95%, or 95-100% identical to one or more CDRs in a light chain variable region sequence set forth as SEQ ID NO:2.

The invention further provides isolated and purified antibodies and functional fragments thereof that have one or more amino acid additions, deletions or substitutions of SEQ ID NO:1 or SEQ ID NO:2. In particular aspects, an antibody or functional fragment has sequence at least 80-85%, 85-90%, 90-95%, or 95-100% identical to a heavy chain variable region sequence set forth as SEQ ID NO:1, or a sequence at least 80-85%, 85-90%, 90-95%, or 95-100% identical to a light chain variable region sequence set forth as SEQ ID NO:2. In further aspects, an antibody or functional fragment has a heavy or light chain sequence with 100% identity to one or more CDRs in a heavy or light chain variable region sequence as represented by DSMZ Deposit No. DSM ACC2859, or set forth as SEQ ID NOs:1 and 2, and has less than 100% identity to a region outside of the CDRs in a heavy or light chain variable region sequence as represented by DSMZ Deposit No. DSM ACC2859, or set forth as SEQ ID NOs:1 and 2.

The invention also provides antibodies and functional fragments thereof that have a binding affinity within about 1-5000 fold of the binding affinity of BARB3 antibody, as represented by DSMZ Deposit No. DSM ACC2859, or heavy and light chain sequences set forth as SEQ ID NOs:1 and 2, for binding to an antigen or a cell (e.g., a neplastic, cancer, tumor or metastatic cell). In various embodiments, antibodies and functional fragments have a binding affinity within about 1-5000 fold of the binding affinity of BARB3 antibody (as represented by DSMZ Deposit No. DSM ACC2859, or heavy and light chain sequences set forth as SEQ ID NOs:1 and 2) for binding to an adenocarcinoma cell or a squamous cell carcinoma, such as a stomach adenocarcinoma cell, a lung adenocarcinoma cell, a pancreas adenocarcinoma cell, a colon adenocarcinoma cell, a breast adenocarcinoma cell, or an esophagus squamous cell carcinoma. In additional embodiments, an antibody or functional fragment has a binding affinity within about 1-5000 fold of the binding affinity of BARB3 antibody (as represented by DSMZ Deposit No. DSM ACC2859, or heavy and light chain sequences set forth as SEQ ID NOs:1 and 2) for binding to a human adenocarcinoma, squamous cell carcinoma, carcinoid carcinoma, invasive ductal carcinoma, germ cell carcinoma of stomach, lung, colon, pancreas, esophagus, prostate, breast or testis. In further embodiments, an antibody or functional fragment has a binding affinity within about 1-5000 fold of the binding affinity of BARB3 antibody (as represented by DSMZ Deposit No. DSM ACC2859, or heavy and light chain sequences set forth as SEQ ID NOs:1 and 2) for binding to a pancreas cancer cell (e.g., BXPC-3 cells), or a stomach cancer cell (e.g., 23132/87 cells). In still further embodiments, an antibody or functional fragment has a binding affinity within about KD 10⁻⁵ M to about KD 10⁻¹³ M for binding to one or more cells or cell lines set forth herein (e.g., an adenocarcinoma cell, a squamous cell carcinoma, a stomach adenocarcinoma cell, a lung adenocarcinoma cell, a pancreas adenocarcinoma cell, a colon adenocarcinoma cell, a breast adenocarcinoma cell, an esophagus squamous cell carcinoma, etc.).

The invention additionally provides antibodies and functional fragments thereof that bind to a cell or cell line that expresses a cell membrane bound prohibitin isoform, as well as antibodies and functional fragments thereof that bind to a cell membrane bound prohibitin isoform antigen. Antibodies and functional fragments thereof moreover provided include those that compete with BARB3 antibody, as represented by DSMZ Deposit No. DSM ACC2859, or heavy and light chain sequences set forth as SEQ ID NOs:1 and 2, for binding to prohibitin (e.g., a cell membrane bound prohibitin isoform).

Antibodies of the invention include IgG, IgA, IgM, IgE and IgD. In various aspects, an IgG is an IgG1, IgG2, IgG3, or IgG4.

Antibody functional fragments and subsequences of the invention include functional fragments and subsequences of the various antibodies set forth herein. In a particular embodiment, a functional fragment of BARB3 antibody (as represented by DSMZ Deposit No. DSM ACC2859, or heavy and light chain sequences set forth as SEQ ID NOs:1 and 2) that competes with BARB3 antibody, as represented by DSMZ Deposit No. DSM ACC2859, or heavy and light chain sequences set forth as SEQ ID NOs:1 and 2, for binding to a cell or antigen, or that retains at least partial binding to a cell or antigen to which BARB3, as represented by DSMZ Deposit No. DSM ACC2859, or heavy and light chain sequences set forth as SEQ ID NOs:1 and 2, binds, is provided. In particular aspects, a functional fragment or a subsequence is an Fab, Fab′, F(ab′)₂, Fv, Fd, single-chain Fv (scFv), disulfide-linked Fvs (sdFv), V_{L}, V_{H}, trispecific (Fab₃), bispecific (Fab₂), diabody ((V_{L}-V_{H})₂ or (V_{H}-V_{L})₂), triabody (trivalent), tetrabody (tetravalent), minibody ((scFᵥ-C_{H}3)₂), bispecific single-chain Fv (Bis-scFv), IgGdeltaCH2, scFv-Fc or (scFv)₂-Fc. In further aspects, a functional fragment or a subsequence of a full length antibody heavy or light chain, or a heavy or light chain variable region, has a length from about 20-30, 30-50, 50-100, 100-150, 150-200, 200-250, 250-300, 300-400, 400-500, amino acid residues.

The invention also provides antibodies and subsequences that include a heterologous domain. In one embodiment, a heterologous domain includes a detectable label, tag or cytotoxic agent. In particular aspects, a detectable label or tag is an enzyme, enzyme substrate, ligand, receptor, radionuclide, a T7-, His-, myc-, HA- or FLAG-tag, electron-dense reagent, energy transfer molecule, paramagnetic label, fluorophore, chromophore, chemi-luminescent agent, or a bio-luminescent agent.

The invention moreover provides nucleic acid sequences that encode antibodies and functional fragments thereof. In one embodiment, a nucleic acid sequence is at least 75-100% complementary or identical to a nucleic acid sequence that encodes a heavy or a light chain variable region sequence of BARB3 antibody, as represented by DSMZ Deposit No. DSM ACC2859, or heavy and light chain sequences set forth as SEQ ID NOs:1 and 2, or a subsequence thereof. In another embodiment, a nucleic acid encodes a subsequence of SEQ ID NOs:1 or 2. In particular aspects, a nucleic acid sequence has a length from about 10-26, 20-30, 30-50, 50-100, 100-150, 150-200, 200-250, 250-300, 300-400, 400-500, or 500-1000 nucleotides. In additional aspects, a nucleic acid sequence specifically hybridizes to a nucleic acid that encodes SEQ ID NO:1, or a subsequence thereof, or specifically hybridizes to a nucleic acid sequence complementary to a nucleic acid that encodes SEQ ID NO:2, or a subsequence thereof. In further aspects, a nucleic acid is an antisense polynucleotide, a small interfering RNA, or a ribozyme nucleic acid that specifically hybridizes to a nucleic acid sequence encoding SEQ ID NO:1, 2 or 5. Antisense polynucleotides, small interfering RNA, and ribozyme polynucleotides can have a length from about 10-20, 20-30, 30-50, 50-100, 100-150, 150-200, 200-250, 250-300, 300-400, 400-500, 500-1000, 1000-2000 nucleotides, and be at least 90% complementary or identical to a nucleic acid sequence that encodes SEQ ID NOs:1, 2 or 5, or a subsequence thereof. In still further aspects, nucleic acid sequence can include an expression control sequence or a vector (e.g., a viral, bacterial, fungal or mammalian vector).

The invention additionally provides isolated and purified cells as well as transformed host cells that express an antibody or subsequence thereof that includes a sequence at least 60% or more (e.g., 65%, 70%, 75%, 80%, 85%, 90%, 95%, etc.) identical to a heavy chain variable region sequence set forth as SEQ ID NO:1, or a sequence at least 60% or more (e.g., 65%, 70%, 75%, 80%, 85%, 90%, 95%, etc.) identical to a light chain variable region sequence of BARB3 antibody, as represented by DSMZ Deposit No. DSM ACC2859, or heavy and light chain sequences set forth as SEQ ID NOs:1 and 2. Such cells include eukaryotic and non-eukaryotic cells, which can stably or transiently express antibody or subsequence thereof, or be stably or transiently transformed with the nucleic acid or vector that encodes antibody or subsequence thereof or.

The invention further provides kits. In various embodiments, a kit includes an antibody or functional fragment thereof that competes with BARB3 antibody, as represented by DSMZ Deposit No. DSM ACC2859, or heavy and light chain sequences set forth as SEQ ID NOs:1 and 2, for binding to an antigen or to a cell (e.g., a neoplastic, cancer, tumor or metastatic cell). In particular aspects, a kit includes an antibody or functional fragment thereof that competes with BARB3 antibody, as represented by DSMZ Deposit No. DSM ACC2859, or heavy and light chain sequences set forth as SEQ ID NOs:1 and 2, for binding to an adenocarcinoma cell or a squamous cell carcinoma, such as a stomach adenocarcinoma cell, a lung adenocarcinoma cell, a pancreas adenocarcinoma cell, a colon adenocarcinoma cell, a breast adenocarcinoma cell, or an esophagus squamous cell carcinoma. In an additional embodiment, a kit includes an antibody or functional fragment thereof that competes with BARB3 antibody, as represented by DSMZ Deposit No. DSM ACC2859, or heavy and light chain sequences set forth as SEQ ID NOs:1 and 2, for binding to a pancreas cancer cell (e.g., BXPC-3 cells), or a stomach cancer cell (e.g., 23132/87 cells).

Kits of the invention also include antibodies and functional fragments that bind to cells or an antigen that BARB3 antibody, as represented by DSMZ Deposit No. DSM ACC2859, or heavy and light chain sequences set forth as SEQ ID NOs:1 and 2, binds. In one embodiment, a kit includes an antibody or functional fragment that binds to an adenocarcinoma cell or a squamous cell carcinoma to which BARB3 antibody, as represented by DSMZ Deposit No. DSM ACC2859, or heavy and light chain sequences set forth as SEQ ID NOs:1 and 2, binds, such as a stomach adenocarcinoma cell, a lung adenocarcinoma cell, a pancreas adenocarcinoma cell, a colon adenocarcinoma cell, a breast adenocarcinoma cell, an esophagus squamous cell carcinoma, to which BARB3 antibody, as represented by DSMZ Deposit No. DSM ACC2859, or heavy and light chain sequences set forth as SEQ ID NOs:1 and 2, binds. In another embodiment, a kit includes an antibody or functional fragment binds to a human adenocarcinoma, squamous cell carcinoma, carcinoid carcinoma, ivasive ductal carcinoma, germ cell carcinoma of stomach, lung, colon, pancreas, esophagus, prostate, breast or testis to which BARB3 antibody, as represented by DSMZ Deposit No. DSM ACC2859, or heavy and light chain sequences set forth as SEQ ID NOs:1 and 2, binds. In an additional embodiment, a kit includes an antibody or functional fragment binds to a pancreas cancer cell (e.g., BXPC-3 cells), a stomach cancer cell (e.g., 23132/87 cells) that BARB3 antibody, as represented by DSMZ Deposit No. DSM ACC2859, or heavy and light chain sequences set forth as SEQ ID NOs:1 and 2, binds.

Kits of the invention further include antibodies and functional fragments that include a heavy or light chain variable region sequence with about 60% or more identity to a heavy or light chain sequence variable regions of BARB3 antibody, as represented by DSMZ Deposit No. DSM ACC2859, or heavy and light chain sequences set forth as SEQ ID NOs:1 and 2. In one embodiment, a kit includes an antibody or subsequence thereof with a sequence at least 60% or more (e.g., 65%, 70%, 75%, 80%, 85%, 90%, 95%, etc.) identical to a heavy chain variable region sequence set forth as SEQ ID NO:1, or to a sequence at least 60% or more (e.g., 65%, 70%, 75%, 80%, 85%, 90%, 95%, etc.) identical to a light chain variable region sequence set forth as SEQ ID NO:2. In another embodiment, a kit includes an antibody or subsequence with a sequence at least 60% or more (e.g., 65%, 70%, 75%, 80%, 85%, 90%, 95%, etc.) identical to a heavy chain variable region sequence set forth as SEQ ID NO:1, and to a sequence at least 60% or more (e.g., 65%, 70%, 75%, 80%, 85%, 90%, 95%, etc.) identical to a light chain variable region sequence set forth as SEQ ID NO:2. In further embodiments, a kit includes an antibody or subsequence with a sequence at least 80-85%, 85-90%, 90-95%, 95-100% identical to one or more CDRs in heavy chain variable region sequence set forth as SEQ ID NO:1, or a sequence at least 80-85%, 85-90%, 90-95%, 95-100% identical to one or more CDRs in a light chain variable region sequence set forth as SEQ ID NO:2.

In additional embodiments, a kit also includes an anti-cell proliferative or immune enhancing treatment or therapeutic agent, or an anti-neoplastic, anti-cancer or anti-tumor agent, or an article of manufacture (e.g., for delivering the antibody, anti-cell proliferative or immune enhancing treatment or therapy into a subject locally, regionally or systemically). In particular aspects, the instructions are for treating undesirable cell proliferation or a cell proliferative disorder (e.g., a neoplasia, tumor cancer or metastasis.

The invention yet additionally provides pharmaceutical compositions. In one embodiment, a composition includes an antibody or functional fragment and a pharmaceutically acceptable carrier or excipient. In another embodiment, a composition includes an antibody that competes with BARB3 antibody, as represented by DSMZ Deposit No. DSM ACC2859, or heavy and light chain sequences set forth as SEQ ID NOs:1 and 2, for binding to a cell or antigen, or that binds to a cell or antigen to which BARB3 antibody binds, or that includes a heavy or light chain variable region sequence with about 60% or more identity to a heavy or light chain sequence variable regions as set forth in SEQ ID NOs:1 or 2 or a sequence at least 80-85%, 85-90%, 90-95%, 95-100% identical to one or more CDRs in a heavy chain or light chain variable region sequence set forth as SEQ ID NO:1 or 2, and a pharmaceutically acceptable carrier or excipient.

Antibodies, functional fragments and modified forms are useful for treating a subject in need of treatment. The invention therefore provides methods of using antibodies and functional fragments in treatment (e.g., therapeutic or prophylactic) of a subject having or at risk of having undesirable cell proliferation, such as a cell proliferative or hyperproliferative disorder. In one embodiment, a method includes administering an antibody or functional fragment (e.g., a BARB3 antibody, as represented by DSMZ Deposit No. DSM ACC2859, or heavy and light chain sequences set forth as SEQ ID NOs:1 and 2) to a subject having or at risk of having undesirable cell proliferation (e.g., a cell proliferative disorder) an amount effective to treat undesirable cell proliferation. In particular aspects, a cell proliferative disorder is a metastatic or non-metastatic, solid or liquid neoplasia, malignancy, tumor or cancer. In further aspects, undesirable cell proliferation (e.g., a cell proliferative disorder) affects or is present at least in part in brain, head or neck, breast, esophagus, mouth, nasopharynx, nose or sinuses, stomach, duodenum, ileum, jejunum, lung, liver, pancreas, kidney, adrenal gland, thyroid, bladder, colon, rectum, prostate, uterus, cervix, ovary, bone marrow, lymph, blood, bone, testes, skin or muscle, or hematopoetic system. In additional aspects, undesirable cell proliferation (e.g., a cell proliferative disorder) includes a neoplasia, tumor, cancer or metastasis that affects or is at least in part present in breast, lung, thyroid, head and neck, nasopharynx, nose or sinuses, brain, spine, adrenal gland, thyroid, lymph, gastrointestinal tract, mouth, esophagus, stomach, duodenum, ileum, jejunum, small intestine, colon, rectum, genito-urinary tract, uterus, ovary, cervix, bladder, testicle, penis, prostate, kidney, pancreas, adrenal gland, liver, bone, bone marrow, lymph, blood, muscle, skin or is hematopoetic. In further particular aspects, a neoplasia, tumor, cancer or metastasis is a sarcoma, carcinoma, adenocarcinoma, melanoma, myeloma, blastoma, glioma, lymphoma leukemia. In additional particular aspects, a neoplasia, tumor or cancer is a lung adenocarcinoma, lung carcinoma, diffuse or interstitial gastric carcinoma, colon adenocarcinoma, prostate adenocarcinoma, esophagus carcinoma, breast carcinoma, pancreas adenocarcinoma, ovarian adenocarcinoma, or uterine adenocarcinoma, or a metastasis thereof.

In another embodiment, a method includes administering an antibody or functional fragment (e.g., a BARB3 antibody, as represented by DSMZ Deposit No. DSM ACC2859, or heavy and light chain sequences set forth as SEQ ID NOs:1 and 2) to a subject having or at risk of having a metastasis an amount effective to reduce or inhibit spread or dissemination of a tumor, cancer or neoplasia to other sites, locations or regions within the subject. In various aspects, a method reduces or inhibits metastasis of a primary tumor or cancer to one or more other sites, the formation or establishment of a metastasis at one or more other sites, thereby inhibiting or reducing tumor or cancer relapse or tumor or cancer progression. In further aspects, a method reduces or inhibits growth, proliferation, mobility or invasiveness of tumor or cancer cells that potentially or do develop metastases (e.g, disseminated tumor cells); reduces or inhibits formation or establishment of metastases arising from a primary tumor or cancer to one or more other sites, locations or regions distinct from the primary tumor or cancer; reduces or inhibits growth or proliferation of a metastasis at one or more other sites, locations or regions distinct from the primary tumor or cancer after the metastasis has formed or has been established; or reduces or inhibits formation or establishment of additional metastasis after the metastasis has been formed or established.

In further particular aspects, a neoplasia, tumor or cancer, or metastasis is progressively worsening or is in remission. In still additional aspects, treatment results in alleviating or ameliorating one or more adverse physical symptoms associated with a cell proliferative disorder, or a neoplasia, tumor or cancer, or reduces or decreases neoplasia, tumor or cancer volume, inhibits or prevents an increase in neoplasia, tumor or cancer volume, inhibits neoplasia, tumor or cancer progression or worsening, stimulates neoplasia, tumor or cancer cell lysis or apoptosis, or inhibits, reduces or decreases neoplasia, tumor or cancer proliferation or metastasis, or prolongs or extends lifespan of the subject, or improves the quality of life of the subject.

Methods include administration to a subject locally, regionally, or systemically. Exemplary subjects (e.g., mammals such as humans) include candidates for, and those undergoing, or having undergone an anti-cell proliferative or anti-hyperproliferative disorder (e.g., anti-neoplastic, anti-tumor, anti-cancer or anti-metastasis) or immune-enhancing treatment or therapy.

The invention yet also provides combined methods for treating a disorder in a subject in need of treatment. In one embodiment, a method includes administering to a subject an antibody that competes with BARB3 antibody, as represented by DSMZ Deposit No. DSM ACC2859, or heavy and light chain sequences set forth as SEQ ID NOs:1 and 2, for binding of to a cell, or binds to a cell to which BARB3 antibody, as represented by DSMZ Deposit No. DSM ACC2859, or heavy and light chain sequences set forth as SEQ ID NOs:1 and 2, binds and an anti-cell proliferative or immune-enhancing treatment or therapy to a subject (e.g., prior to, substantially contemporaneously with or following each other). In various aspects, an anti-cell proliferative or immune-enhancing treatment or therapy includes surgical resection, radiotherapy, radiation therapy, chemotherapy, immunotherapy, hyperthermia, an alkylating agent, anti-metabolite, plant extract, plant alkaloid, nitrosourea, hormone, nucleoside or nucleotide analogue, a lymphocyte, plasma cell, macrophage, dendritic cell, NK cell or B-cell, an antibody, a cell growth factor, a cell survival factor, a cell differentiative factor, a cytokine or a chemokine.

Antibodies and functional fragments thereof are useful for detecting, screening for and identifying the presence of cells or antigen (e.g., prohibitin, such as a cell membrane bound prohibitin isoform) that bind to BARB3 antibody, as represented by DSMZ Deposit No. DSM ACC2859, or heavy and light chain sequences set forth as SEQ ID NOs:1 and 2, or antigen that binds to BARB3 antibody, as represented by DSMZ Deposit No. DSM ACC2859, or heavy and light chain sequences set forth as SEQ ID NOs:1 and 2. The invention therefore provides methods for detecting or screening for cells and antigens that bind to BARB3 antibody, as represented by DSMZ Deposit No. DSM ACC2859, or heavy and light chain sequences set forth as SEQ ID NOs:1 and 2, methods for identifying a subject that is amenable to treatment in accordance with the methods of the invention. In one embodiment, a method includes contacting a biological material or sample with an antibody or functional fragment under conditions allowing binding between antibody or functional fragment and cell or antigen that binds to BARB3 antibody, as represented by DSMZ Deposit No. DSM ACC2859, or heavy and light chain sequences set forth as SEQ ID NOs:1 and 2, and assaying for binding of the antibody or functional fragment to a cell or antigen that binds to BARB3 antibody, as represented by DSMZ Deposit No. DSM ACC2859, or heavy and light chain sequences set forth as SEQ ID NOs:1 and 2. The binding of the antibody or functional fragment to a cell or antigen that binds to BARB3 antibody, as represented by DSMZ Deposit No. DSM ACC2859, or heavy and light chain sequences set forth as SEQ ID NOs:1 and 2, indicates that the biological material contains the cell or antigen that binds to BARB3 antibody, as represented by DSMZ Deposit No. DSM ACC2859, or heavy and light chain sequences set forth as SEQ ID NOs:1 and 2. In one aspect, the biological material or sample is obtained from a mammalian (e.g., primate, such as a human) subject.

The invention moreoever provides methods for diagnosing a subject having or at increased risk of having undesirable cell proliferation or a cell proliferative disorder (e.g., neoplasia, tumor or cancer, or metastasis). In various embodiments, a method includes providing a biological material or sample from a subject, contacting the biological material or sample with an antibody or functional fragment that competes with BARB3 antibody, as represented by DSMZ Deposit No. DSM ACC2859, or heavy and light chain sequences set forth as SEQ ID NOs:1 and 2, for binding or an antibody or functional fragment that binds to a cell or antigen to which BARB3 antibody, as represented by DSMZ Deposit No. DSM ACC2859, or heavy and light chain sequences set forth as SEQ ID NOs:1 and 2, binds, or an antibody or functional fragment that includes a heavy or light chain variable region sequence with about 60% or more identity to a heavy or light chain sequence variable regions of BARB3 antibody, as represented by DSMZ Deposit No. DSM ACC2859, or heavy and light chain sequences set forth as SEQ ID NOs:1 and 2, under conditions allowing binding of the antibody or functional fragment, and assaying for binding of the antibody to a cell or antigen that binds to BARB3. In particular aspects, the methods for diagnosing a subject identify those that have or are at increased risk of having undesirable cell proliferation or a cell proliferative disorder (e.g., neoplasia, tumor or cancer, or metastasis). In one aspect, the biological material or sample is obtained from a mammalian (e.g., primate, such as a human) subject. In additional aspects, the biological material or sample comprises a biopsy, such as a lung, pancreas, stomach, breast, esophageal, ovarian or uterine biopsy. In further aspects, the cell or cell line expresses prohibitin (e.g., a cell membrane bound prohibitin isoform), or the antigen comprises prohibitin (e.g., a cell membrane bound prohibitin isoform antigen).

### DESCRIPTION OF DRAWINGS

FIGS. 1A and 1B: BARB3 stomach cancer A) cell proliferation inhibitory; and B) apoptosis stimulating activity.

FIG. 2: BARB3 malignant melanoma cancer cell (HTB-69) proliferation inhibitory activity.

FIG. 3: Immunofluorescence of BARB3 showing endocytosis of BARB3 antibody by pancreas carcinoma cells (BXPC-3) 30 minutes after binding.

FIG. 4: shows reduced binding of BARB3 to BXPC-3 cancer cells treated with N-glycosidase, but not O-glycosidase.

FIG. 5: shows CRL 1424 lysate antigen(s) immunoprecipitated with BARB3 antibody (right lane, antibody heavy and light chain indicated), compared to various controls. Horizontal arrows 1, 2 and 3 in the BARB3 immunoprecipitated lane were subjected to sequence analysis for BARB3 target identification. Angled arrows represent BARB3 antibody degradation products.

### DETAILED DESCRIPTION

The invention is based, at least in part, on antibodies that bind to various neoplastic, cancer, tumor and metastatic cells. A non-limiting exemplary antibody is designated BARB3, as represented by DSMZ Deposit No. DSM ACC2859, or heavy and light chain sequences set forth as SEQ ID NOs:1 and 2. BARB3, as represented by DSMZ Deposit No. DSM ACC2859, or heavy and light chain sequences set forth as SEQ ID NOs:1 and 2, is a human IgG antibody that specifically binds to various neoplastic, cancer, tumor and metastatic cells. BARB3 therefore binds to an antigen expressed on various neoplastic, cancer, tumor and metastatic cells. BARB3 is able to inhibit or reduce proliferation of various neoplastic, cancer, tumor and metastatic cells. BARB3 is also able to stimulate or induce apoptosis of various neoplastic, cancer, tumor and metastatic cells.

Antibodies of the invention include polyclonal and monoclonal antibodies. Antibodies are proteins which include amino acids, or “residues,” covalently linked by an amide bond or equivalent. The term “monoclonal,” when used in reference to an antibody refers to an antibody that is based upon, obtained from or derived from a single clone, including any eukaryotic, prokaryotic, or phage clone. A “monoclonal” antibody is therefore defined herein structurally, and not the method by which it is produced.

Antibodies of the invention can belong to any antibody class, IgM, IgG, IgE, IgA, IgD, or subclass. Exemplary subclasses for IgG are IgG₁, IgG₂, IgG₃ and IgG₄.

Antibodies of the invention can have kappa or lambda light chain sequences, either full length as in naturally occurring antibodies, mixtures thereof (i.e., fusions of kappa and lambda chain sequences), and subsequences/fragments thereof. Naturally occurring antibody molecules contain two kappa or two lambda light chains. The primary difference between kappa and lambda light chains is in the sequences of the constant region.

The amino acid sequences of BARB3, heavy and light chain variable region sequences, as represented by SEQ ID NOs:1 and 2, respectively, are shown.

Amino acid sequence of amplified BARB3 heavy chain (SEQ ID NO:1):

PVLVKATETLTLTCTVSGFSLSNAGMGVSWIRQPPGKALEWLAHIFSND
EKSYSTSLERRLTISKDTSKSQVVLTMTNMDPVDTGTYYCARTRQPYNQ
NMDVWGQGTLVT

BARB3-HC Heavy chain sequence (SEQ ID NO:1, top) and comparison with most homologous germeline gene (M99648 IGHV2-26*01, bottom):

```
<--------------------------------------------- FR1 - IMGT
1 5 10 15
                                                                  P V L V K A
... ... ... ... ... ... ... .gt cct ... gtg ctg gtg aaa gcc
  Q V T L K E S G P
cag gtc acc ttg aag gag tct g-- --- ... --- --- --- --- c--
------------------------------------------> _{———————————————————}
                                  20 25 30
  T E T L T L T C T V S G F S L
aca gag acc ctc acg ctg acc tgc acc gtc tct ggg ttc tca ctc
--- --- --- --- --- --- --- --- --- --- --- --- --- --- ---
_{——} CDR1 - IMGT _{—————————————————} <---------------------------
                                35 40 45
  S N A G M G V S W I R Q P
agc aat gct gga atg ggt ... ... gtg agc tgg atc cgt cag ccc
                          R
--- --- --- a-- --- --- ... ... --- --- --- --- --- --- ---
FR2 - IMGT -------------------------> _{—————————————————} CDR2
                              50 55 60
  P G K A L E W L A H I F S N D
cca ggg aag gcc ctg gag tgg ctt gca cac att ttt tcg aat gac
--- --- --- --- --- --- --- --- --- --- --- --- --- --- ---
- IMGT _{———————————} <---------------------------------------
                              65 70 75
  E K S Y S T S L E R R
gaa aaa ... ... ... tcc tac agc aca tct ctg gag ... aga aga
                                                                                          K S
--- --- ... ... ... --- --- --- --- --- --- a-- ... --c --g
------------------------------- FR3 - IMGT --------------
                                80 85 90
  L T I S K D T S K S Q V V L T
ctc acc atc tcc aag gac acc tcc aaa agc cag gtg gtc ctt acc
--- --- --- --- --- --- --- --- --- --- --- --- --- --- ---
------------------------------------------------------> _{———}
                                95 100 104
  M T N M D P V D T G T Y Y C A
atg acc aac atg gac ccc gtg gac aca ggc aca tat tat tgt gca
                                                                          A
--- --- --- --- --- --t --- --- --- -c- --- --- --c --- ---
_{———————————————} CDR3 - IMGT _{————————————————————————————}
  R T R Q P Y N Q N M D V W G Q
cgg acc cgt cag ccg tat aac cag aat atg gac gtc tgg ggc caa
          I
--- -ta -
  G T L V T
gga acc ctg gtc acc c
```

BARB3 heavy chain CDR3 (by IMGT):

gcacggacccgtcagccgtataaccagaatatggacgtc
A R T R Q P Y N Q N M D V

DNA Sequence of amplified BARB3 heavy chain (SEQ ID NO:3):

GGGTGACCAGGGTTCCTTGGCCCCAGACGTCCATATTCTGGTTATACG
GCTGACGGGTCCGTGCACAATAATATGTGCCTGTGTCCACGGGGTCCA
TGTTGGTCATGGTAAGGACCACCTGGCTTTTGGAGGTGTCCTTGGAGA
TGGTGAGTCTTCTCTCCAGAGATGTGCTGTAGGATTTTTCGTCATTCGA
AAAAATGTGTGCAAGCCACTCCAGGGCCTTCCCTGGGGGCTGACGGGAT
CCAGCTCACACCCATTCCAGCATTGCTGAGTGAGAACCCAGAGACGGT
GCAGGTCAGCGTGAGGGTCTCTGTGGCTTTCACCAGCACAGGAC

Amino Acid Sequence of amplified BARB3 light chain (SEQ ID NO:2):

QTVVTQEPSFSVSPGGTVTLTCALSSGSVSPSYYPSWSQQTPGQAPRTLI
YSTNIRSSGVPDRFSGSVLGNKAALTITGAQAEDESDYYCMLYVGSAIWV
FGGGTK

BARB3-LC Light chain sequence (SEQ ID NO:2, top) and comparison with most homologous germeline gene (Z73650 IGLV8-61*01, bottom):

```
<--------------------------------------------- FR1 - IMGT
1 5 10 15
  Q T V V T Q E P S F S V S P
cag act gtg gtg acc cag gag cca tcg ... ttc tca gtg tcc cct
--- --- --- --- --- --- --- --- --- ... --- --- --- --- ---
------------------------------------------> _{———————————————————}
                                20 25 30
  G G T V T L T C A L S S G S V
gga ggg aca gtc aca ctc act tgt gcc ttg agc tct ggc tca gtc
                                                                  G
--- --- --- --- --- --- --- --- -g- --- --- --- --- --- ---
_{——} CDR1 - IMGT _{———————————————————} <--------------------------
                                35 40 45
  S P S Y Y P S W S Q Q T
tct cct agt tat tac ... ... ... ccc agc tgg tcc cag cag act
          T Y
--- a-- --- --c --- ... ... ... --- --- --- -a- --- --- --c
FR2 - IMGT -------------------------> _{——————————————————} CDR2
                                50 55 60
  P G Q A P R T L I Y S T N
cca ggc cag gct cca cgc aca ctc atc tac agc aca aat ... ...
--- --- --- --- --- --- --g --- --- --- --- --- --c ... ...
- IMGT _{—————————————} <---------------------------------------
                              65 70 75
                                          I R S S G V P D R
... ... ... ... ... att cgc tct tct ggg gtc cct ... gat cgc
                                          T
... ... ... ... ... -c- --- --- --- --- --- --- ... --- ---
------------------------------- FR3 - IMGT --------------
                                80 85 90
  F S G S V L G N K A A L T
ttc tct ggc tcc gtc ... ... ctt ggg aac aaa gct gcc ctc acc
                                  I
--- --- --- --- a-- ... ... --- --- --- --- --- --- --- ---
------------------------------------------------------> _{———}
                                95 100 104
  I T G A Q A E D E S D Y Y C M
atc acg ggg gcc cag gct gaa gat gag tct gat tat tat tgt atg
                                                  D V
--- --- --- --- --- --a --t --- --a --- --- --- --c --- g--
_{————————————} CDR3 - IMGT _{—————————————}
  L Y V G S A I W V F G G G T K
ttg tat gtg ggt agt gcc att tgg gtg ttc ggc gga ggg acc aag
                  M G
c-- --- a-- --- --- -g- --- -c
```

BARB3 light chain CDR3 (by IMGT):

atg ttg tat gtg ggt agt gcc att tgg gtg
M L Y V G S A I W V

DNA Sequence of amplified BARB3 light chain (SEQ ID NO:4):

CAGACTGTGGTGACCCAGGAGCCATCGTTCTCAGTGTCCCCTGGAGGG
ACAGTCACACTCACTTGTGCCTTGAGCTCTGGCTCAGTCTCTCCTAGTT
ATTACCCCAGCTGGTCCCAGCAGACTCCAGGCCAGGCTCCACGCACAC
TCATCTACAGCACAAATATTCGCTCTTCTGGGGTCCCTGATCGCTTCTC
TGGCTCCGTCCTTGGGAACAAAGCTGCCCTCACCATCACGGGGGCCCA
GGCTGAAGATGAGTCTGATTATTATTGTATGTTGTATGTGGGTAGTGC
CATTTGGGTGTTCGGCGGAGGGACCAAG

Predicted CDRs, of which there are three in each of heavy and light chain sequence set forth as SEQ ID NOs:1 and 2, are conveniently denoted herein as LC-CDR1, LC-CDR2 and LC-CDR3; and HC-CDR1, HC-CDR2 and HC-CDR3. The CDR sequences of each of SEQ ID NOs:1 and 2 were determined by alignments with homologous germline sequences. In particular, to determine the positions of the CDRs, IMGT (International ImMunoGeneTics Information System) and MRC (VBASE, MRC Centre for Protein Engineering) sequence databases were used. Typically, the IMGT database gives shorter CDR regions for lambda light chain.

Based upon the IMGT database, BARB3 Heavy Chain CDR1, GFSLSNAGMG; CDR2, IFSNDEK; and CDR3, ARTRQPYNQNMDV.

Based upon the IMGT database, BARB3 Light Chain CDR1, SGSVSPSYY; CDR2, STN; and CDR3, MLYVGSAIWV. Based upon the MRC database, BARB3 Light Chain CDR1, ALSSGSVSPSYYPS; CDR2, STNIRSS; and CDR3, MLYVGSAIW.

The location of additional regions, such as D- and J-regions are also known to the skilled artisan.

In accordance with the invention, there are provided isolated and purified antibodies and functional (e.g., cell or antigen binding) fragments structurally and/or functionally related to BARB3, as represented by DSMZ Deposit No. DSM ACC2859, or heavy and light chain variable region sequences set forth as SEQ ID NOs:1 and 2, respectively. In various embodiments, antibodies and functional fragments compete with BARB3 antibody, as represented by DSMZ Deposit No. DSM ACC2859, or heavy and light chain variable region sequences set forth as SEQ ID NOs:1 and 2, for binding to a cell or antigen. In additional embodiments, antibodies and functional fragments compete with BARB3 antibody, as represented by DSMZ Deposit No. DSM ACC2859, or heavy and light chain variable region sequences set forth as SEQ ID NOs:1 and 2, for binding of to an adenocarcinoma cell or a squamous cell carcinoma. In further embodiments, antibodies and functional fragments compete with BARB3 antibody, as represented by DSMZ Deposit No. DSM ACC2859, or heavy and light chain variable region sequences set forth as SEQ ID NOs:1 and 2, for binding of to one or more of a stomach adenocarcinoma cell, a lung adenocarcinoma cell, a pancreas adenocarcinoma cell, a colon adenocarcinoma cell, a breast adenocarcinoma cell, or an esophagus squamous cell carcinoma. In yet additional embodiments, antibodies and functional fragments compete with BARB3 antibody, as represented by DSMZ Deposit No. DSM ACC2859, or heavy and light chain variable region sequences set forth as SEQ ID NOs:1 and 2, for binding to a human adenocarcinoma, human squamous cell carcinoma, human carcinoid carcinoma, human invasive ductal carcinoma, or a human germ cell carcinoma of any of stomach, lung, colon, pancreas, esophagus, prostate, breast or testis. In still further embodiments, antibodies and functional fragments compete with BARB3 antibody, as represented by DSMZ Deposit No. DSM ACC2859, or heavy and light chain variable region sequences set forth as SEQ ID NOs:1 and 2, for binding of to a pancreas cancer cell line BXPC-3 (ATCC Deposit No. CRL-1687) or a stomach cancer cell line 23132/87 (DSMZ Deposit No. ACC 201). In particular aspects, antibodies and functional fragments competitively inhibit binding of BARB3 antibody, as represented by DSMZ Deposit No. DSM ACC2859, or heavy and light chain variable region sequences set forth as SEQ ID NOs:1 and 2, to a cell or antigen by at least 30%, 40%, 50%, 60%, 70%, 80%, 90% or more.

In accordance with the invention, there are also provided antibodies and functional fragments that bind to a cell or antigen that BARB3 antibody, as represented by DSMZ Deposit No. DSM ACC2859, or heavy and light chain variable region sequences set forth as SEQ ID NOs:1 and 2, binds. In one embodiment, an isolated or purified antibody or functional fragment thereof binds to a cell or antigen that BARB3 antibody, as represented by DSMZ Deposit No. DSM ACC2859, or heavy and light chain variable region sequences set forth as SEQ ID NOs:1 and 2, binds. In particular aspects, the antibody or functional fragment thereof binds to a cell or antigen present on an adenocarcinoma cell or a squamous cell carcinoma to which BARB3 antibody, as represented by DSMZ Deposit No. DSM ACC2859, or heavy and light chain variable region sequences set forth as SEQ ID NOs:1 and 2, binds. In additional particular aspects, the antibody or functional fragment thereof binds to a cell or antigen present on one or more of a stomach adenocarcinoma cell, a lung adenocarcinoma cell, a pancreas adenocarcinoma cell, a colon adenocarcinoma cell, a breast adenocarcinoma cell, an esophagus squamous cell carcinoma, to which BARB3 antibody, as represented by DSMZ Deposit No. DSM ACC2859, or heavy and light chain variable region sequences set forth as SEQ ID NOs:1 and 2, binds. In further particular aspects, the antibody or functional fragment thereof binds to a cell or antigen present on a human adenocarcinoma, squamous cell carcinoma, carcinoid carcinoma, invasive ductal carcinoma, germ cell carcinoma of stomach, lung, colon, pancreas, esophagus, prostate, breast or testis to which intact BARB3 antibody, as represented by DSMZ Deposit No. DSM ACC2859, or heavy and light chain variable region sequences set forth as SEQ ID NOs:1 and 2, binds. In still further particular aspects, the antibody or functional fragment thereof binds to a cell or antigen present on a pancreas cancer cell line BXPC-3 (ATCC Deposit No. CRL-1687) or a stomach cancer cell line 23132/87 (DSMZ Deposit No. ACC 201) that intact BARB3 antibody, as represented by DSMZ Deposit No. DSM ACC2859, or heavy and light chain sequences set forth as SEQ ID NOs:1 and 2, binds.

Cells (e.g., neoplastic, cancer, tumor or metastatic cells), cell lines (e.g., neoplastic, cancer, tumor or metastatic cell lines) or antigen to which antibodies bind include cells and cell lines that express a cell membrane isoform of prohibitin and an antigen comprising a cell membrane isoform of prohibitin. A representative human prohibitin sequence (SEQ ID NO:5) is:

MAAKVFESIGKFGLALAVAGGVVNSALYNVDAGHRAVIFDRFRGVQDIV
VGEGTHFLIPWVQKPIIFDCRSRPRNVPVITGSKDLQNVNITLRILFRP
VASQLPRIFTSIGEDYDERVLPSITTEILKSVVARFDAGELITQRELVS
RQVSDDLTERAATFGLILDDVSLTHLTFGKEFTEAVEAKQVAQQEAERA
RFVVEKAEQQKKAAIISAEGDSKAAELIANSLATAGDGLIELRKLEAAE
DIAYQLSRSRNITYLPAGQSVLLQLPQ

The invention therefore further provides antibodies and functional fragments thereof that bind to a cell or cell line that expresses a cell membrane bound prohibitin isoform, as well as antibodies and functional fragments thereof that bind to a cell membrane bound prohibitin isoform antigen. Antibodies and functional fragments thereof moreover provided include those that compete with BARB3 antibody, as represented by DSMZ Deposit No. DSM ACC2859, or heavy and light chain sequences set forth as SEQ ID NOs:1 and 2, for binding to a prohibitin sequence (e.g., a cell membrane bound prohibitin isoform, or SEQ ID NO:5).

The term “bind,” or “binding,” when used in reference to an antibody or functional fragment, means that the antibody or functional fragment interacts at the molecular level with a corresponding epitope (antigenic determinant) present on a cell or an antigen. Epitopes of antigens that comprise amino acids typically include relatively short sequences, e.g. about five to 15 amino acids in length. Epitopes can be contiguous or non-contiguous. A non-contiguous amino acid sequence epitope forms due to protein folding. Techniques for identifying epitopes are known to the skilled artisan and include screening overlapping oligopeptides for binding to antibody (for example, U.S. Pat. No. 4,708,871), phage display peptide library kits, which are commercially available for epitope mapping (New England BioLabs). Epitopes may also be identified by inference when epitope length peptide sequences are used to immunize animals from which antibodies that bind to the peptide sequence are obtained and can be predicted using computer programs, such as BEPITOPE (Odorico et al., J. Mol. Recognit. 16:20 (2003)).

The invention further provides antibodies and functional fragments that inhibit, decrease or reduce cell growth or proliferation, or stimulate or induce cell death, lysis or apoptosis. In particular embodiments, binding of BARB3 antibody, as represented by DSMZ Deposit No. DSM ACC2859, or heavy and light chain variable region sequences set forth as SEQ ID NOs:1 and 2, to a neoplastic, tumor or cancer, or metastasis cell inhibits, decreases or reduces cell growth or proliferation, or stimulates or induces cell death, lysis or apoptosis. In another embodiment, binding of BARB3 antibody, as represented by DSMZ Deposit No. DSM ACC2859, or heavy and light chain variable region sequences set forth as SEQ ID NOs:1 and 2, to BXPC-3 or 23132/87 cells inhibits, decreases or reduces cell growth or proliferation, or stimulates or induces cell death, lysis or apoptosis.

The invention moreover provides of antibodies and functional fragments that are structurally and/or functionally related to BARB3 antibody, as represented by DSMZ Deposit No. DSM ACC2859; or heavy and light chain variable region sequences set forth as SEQ ID NOs:1 and 2, in which include the heavy or light chain variable region sequence exhibits a degree of identity to SEQ ID NOs:1 or 2, or exhibits identity to a sequence within SEQ ID NOs:1 or 2 (e.g., one or more CDRs). In particular embodiments, antibodies and functional fragments include a heavy or a light chain variable region sequence with about 60% or more identity to a heavy or light chain sequence variable region of BARB3 antibody, as represented by DSMZ Deposit No. DSM ACC2859, or heavy and light chain variable region sequences set forth as SEQ ID NOs:1 and 2, or a sequence within SEQ ID NOs:1 or 2 (e.g., one or more CDRs). In other particular embodiments, antibodies or functional fragments include a heavy or a light chain with at least 65%, 70%, 75%, 80%, 85%, 90%, 95%, or more identity to a heavy chain variable region sequence of BARB3 antibody, as represented by DSMZ Deposit No. DSM ACC2859, or heavy and light chain variable region sequences set forth as SEQ ID NOs:1 and 2, or a sequence within SEQ ID NOs:1 or 2 (e.g., one or more CDRs). In additional particular embodiments, antibodies or functional fragments include a heavy or a light chain variable region sequence with at least 80-85%, 85-90%, 90-95%, 95-100% identity to one or more CDRs in BARB3 antibody, as represented by DSMZ Deposit No. DSM ACC2859, or heavy and light chain variable region sequences set forth as SEQ ID NOs:1 and 2. In a particular aspect, an antibody or a functional fragment thereof includes a heavy or a light chain variable region sequence with 95-100% identity to one, two or three CDRs in each heavy or light chain variable region sequences in BARB3 antibody, as represented by DSMZ Deposit No. DSM ACC2859, or heavy and light chain variable region sequences set forth as SEQ ID NOs:1 and 2.

Antibodies and functional fragments of the invention therefore include those with at least partial sequence identity to BARB3 antibody, as represented by DSMZ Deposit No. DSM ACC2859, or heavy and light chain variable region sequences set forth as SEQ ID NOs:1 and 2. The percent identity of such antibodies and functional fragments can be as little as 60%, or can be more (e.g., 65%, 70%, 75%, 80%, 85%, 90%, 95%, etc.).

The percent identity can extend over the entire sequence length of BARB3 antibody, as represented by DSMZ Deposit No. DSM ACC2859, or heavy and light chain variable region sequences set forth as SEQ ID NOs:1 and 2, or a contiguous region or area within BARB3 antibody, as represented by DSMZ Deposit No. DSM ACC2859, or heavy and light chain variable region sequences set forth as SEQ ID NOs:1 and 2. In particular aspects, the length of the sequence sharing the percent identity is 5 or more contiguous amino acids, e.g., 5, 6, 7, 8, 9, 10, 11, 12, 13, 14, 15, 16, 17, 18, 19, 20, 21, 22, 23, 24, 25, etc. contiguous amino acids. In additional particular aspects, the length of the sequence sharing the percent identity is 25 or more contiguous amino acids, e.g., 26, 27, 28, 29, 30, 31, 32, 33, 34, 35, etc. contiguous amino acids. In further particular aspects, the length of the sequence sharing the percent identity is 35 or more contiguous amino acids, e.g., 35, 36, 37, 38, 39, 40, 41, 42, 43, 44, 45, 45, 47, 48, 49, 50, etc., contiguous amino acids. In yet additional particular aspects, the length of the sequence sharing the percent identity is 50 or more contiguous amino acids, e.g., 50-55, 55-60, 60-65, 65-70, 70-75, 75-80, 80-85, 85-90, 90-95, 95-100, 100-110, etc. contiguous amino acids. In yet further particular aspects, the length of the sequence sharing the percent identity is equal to the length of any CDR of a variable region sequence, or a region outside the CDRs but within the variable region of a heavy or light chain sequence, such as BARB3 antibody represented by DSMZ Deposit No. DSM ACC2859, or heavy and light chain variable region sequences set forth as SEQ ID NOs:1 and 2.

The term “identity” and grammatical variations thereof, mean that two or more referenced entities are the same. Thus, where two antibody sequences are identical, they have the same amino acid sequence, at least within the referenced region or portion. Where two nucleic acid sequences are identical, they have the same polynucleotide sequence, at least within the referenced region or portion. The identity can be over a defined area (region or domain) of the sequence. An “area of identity” refers to a portion of two or more referenced entities that are the same. Thus, where two protein or nucleic acid sequences are identical over one or more sequence regions they share identity within that region. Exemplary identity are antibodies and functional fragments with an amino acid sequence with 50%, 60%, 70%, 75%, 80%, 85%, 90%, 95%, 96%, 97%, 98%, or more sequence identity to a reference antibody or functional fragment, for example, BARB3 antibody, as represented by DSMZ Deposit No. DSM ACC2859, or heavy and light chain variable region sequences set forth as SEQ ID NOs:1 and 2.

The terms “homologous” or “homology” mean that two or more referenced entities share at least partial identity over a given region or portion. “Areas, regions or domains” of homology or identity mean that a portion of two or more referenced entities share homology or are the same. Thus, where two antibody sequences are identical over one or more sequence regions they share identity in these regions. “Substantial homology” means that a molecule is structurally or functionally conserved such that it has or is predicted to have at least partial structure or function of one or more of the structures or functions (e.g., a biological function) of the reference molecule, or relevant/corresponding region or portion of the reference molecule to which it shares homology. An antibody or functional fragment with substantial homology has or is predicted to have at least partial activity or function as the reference antibody. For example, in a particular embodiment, a BARB3 antibody, as represented by DSMZ Deposit No. DSM ACC2859, or heavy and light chain variable region sequences set forth as SEQ ID NOs:1 and 2, with one or more modifications (e.g., substitutions, deletions or additions of SEQ ID NO:1 or SEQ ID NO:2) retain the ability to at least partially compete for binding of BARB3 antibody, as represented by DSMZ Deposit No. DSM ACC2859, or heavy and light chain variable region sequences set forth as SEQ ID NOs:1 and 2, to a cell or antigen, or at least retains partial binding to a cell or antigen to which BARB3 antibody, as represented by DSMZ Deposit No. DSM ACC2859, or heavy and light chain variable region sequences set forth as SEQ ID NOs:1 and 2, binds is considered to have substantial homology to BARB3 antibody, as represented by DSMZ Deposit No. DSM ACC2859, or heavy and light chain variable region sequences set forth as SEQ ID NOs:1 and 2.

The extent of identity (homology) between two sequences can be ascertained using a computer program and mathematical algorithm known in the art. Such algorithms that calculate percent sequence identity (homology) generally account for sequence gaps and mismatches over the comparison region or area. For example, a BLAST (e.g., BLAST 2.0) search algorithm (see, e.g., Altschul et al., J. Mol. Biol. 215:403 (1990), publicly available through NCBI) has exemplary search parameters as follows: Mismatch-2; gap open 5; gap extension 2. For polypeptide sequence comparisons, a BLASTP algorithm is typically used in combination with a scoring matrix, such as PAM100, PAM 250, BLOSUM 62 or BLOSUM 50. FASTA (e.g., FASTA2 and FASTA3) and SSEARCH sequence comparison programs are also used to quantitate the extent of identity (Pearson et al., Proc. Natl. Acad. Sci. USA 85:2444 (1988); Pearson, Methods Mol. Biol. 132:185 (2000); and Smith et al., J. Mol. Biol. 147:195 (1981)). Programs for quantitating protein structural similarity using Delaunay-based topological mapping have also been developed (Bostick et al., Biochem Biophys Res Commun. 304:320 (2003)).

Antibodies and functional fragments of the invention include those that retain at least one or more partial activities or functions of BARB3 antibody, as represented by DSMZ Deposit No. DSM ACC2859, or heavy and light chain variable region sequences set forth as SEQ ID NOs:1 and 2. As disclosed herein, the antigen to which BARB3 antibody, as represented by DSMZ Deposit No. DSM ACC2859; or heavy and light chain sequences set forth as SEQ ID NOs:1 and 2, binds is expressed on malignant and non-malignant, neoplastic, tumor and cancer cells. Non-limiting examples of cells that bind to BARB3, as represented by DSMZ Deposit No. DSM ACC2859, or heavy and light chain variable region sequences set forth as SEQ ID NOs:1 and 2 light and heavy chain, and therefore express a target antigen of BARB3 include a stomach adenocarcinoma cell, a lung adenocarcinoma cell, a pancreas adenocarcinoma cell, a colon adenocarcinoma cell, a breast adenocarcinoma cell, or an esophagus squamous cell carcinoma, a human adenocarcinoma, human squamous cell carcinoma, human carcinoid carcinoma, human invasive ductal carcinoma, or a human germ cell carcinoma in any of stomach, lung, colon, pancreas, esophagus, prostate, breast or testis, or a pancreas cancer cell line BXPC-3 (ATCC Deposit No. CRL-1687) or a stomach cancer cell line 23132/87 (DSMZ Deposit No. ACC 201). Thus, in various embodiments, an antibody or functional fragment binds to one or more cells, such as a stomach adenocarcinoma cell, a lung adenocarcinoma cell, a pancreas adenocarcinoma cell, a colon adenocarcinoma cell, a breast adenocarcinoma cell, or an esophagus squamous cell carcinoma, a human adenocarcinoma, human squamous cell carcinoma, human carcinoid carcinoma, human invasive ductal carcinoma, or a human germ cell carcinoma in any of stomach, lung, colon, pancreas, esophagus, prostate, breast or testis, or a pancreas cancer cell line BXPC-3 (ATCC Deposit No. CRL-1687) or a stomach cancer cell line 23132/87 (DSMZ Deposit No. ACC 201).

Antibodies and functional fragments that bind to a cell or antigen to which BARB3 antibody, as represented by DSMZ Deposit No. DSM ACC2859, or heavy and light chain variable region sequences set forth as SEQ ID NOs:1 and 2, binds can have greater or less relative binding affinity for a cell or an antigen than BARB3 antibody, as represented by DSMZ Deposit No. DSM ACC2859, or heavy and light chain variable region sequences set forth as SEQ ID NOs:1 and 2. Additional antibodies and functional fragments of the invention therefore include those that have greater than, about the same or less than the binding affinity of BARB3 antibody, as represented by DSMZ Deposit No. DSM ACC2859, or heavy and light chain variable region sequences set forth as SEQ ID NOs:1 and 2 light and heavy chain, for binding to a cell or antigen. For example, an antibody or functional fragment of the invention may have an affinity greater or less than 2-5,5-10, 10-100, 100-1000 or 1000-10,000-fold affinity, or any numerical value or range within or encompassing such values, than BARB3 antibody, as represented by DSMZ Deposit No. DSM ACC2859, or heavy and light chain sequences set forth as SEQ ID NOs:1 and 2. In one embodiment, an antibody or a functional thereof has a binding affinity within about 1-5000 fold of the binding affinity of BARB3 antibody, as represented by DSMZ Deposit No. DSM ACC2859, or heavy and light chain sequences set forth as SEQ ID NOs:1 and 2, for binding to a neoplastic, cancer, tumor or metastatic cell. In another embodiment, an antibody or a functional thereof has a binding affinity within about 1-5000 fold of the binding affinity of BARB3 antibody, as represented by DSMZ Deposit No. DSM ACC2859, or heavy and light chain sequences set forth as SEQ ID NOs:1 and 2, for binding to an adenocarcinoma cell or a squamous cell carcinoma, such as a stomach adenocarcinoma cell, a lung adenocarcinoma cell, a pancreas adenocarcinoma cell, a colon adenocarcinoma cell, a breast adenocarcinoma cell, or an esophagus squamous cell carcinoma. In a further embodiment, an antibody or a functional thereof has a binding affinity within about 1-5000 fold of the binding affinity of BARB3 antibody, as represented by DSMZ Deposit No. DSM ACC2859, or heavy and light chain sequences set forth as SEQ ID NOs:1 and 2, for binding to a human adenocarcinoma, human squamous cell carcinoma, human carcinoid carcinoma, human invasive ductal carcinoma, or human germ cell carcinoma of any of stomach, lung, colon, pancreas, esophagus, prostate, breast or testis. In an additional embodiment, an antibody or a functional thereof has a binding affinity within about 1-5000 fold of the binding affinity of BARB3 antibody, as represented by DSMZ Deposit No. DSM ACC2859, or heavy and light chain sequences set forth as SEQ ID NOs:1 and 2, for binding to pancreas cancer BXPC-3 cells or stomach cancer 23132/87 cells. In the foregoing embodiments binding affinity can be 1-5000 fold greater or less than the binding affinity of BARB3 antibody, as represented by DSMZ Deposit No. DSM ACC2859, or heavy and light chain sequences set forth as SEQ ID NOs:1 and 2.

Binding affinity can be determined by association (Kₐ) and dissociation (K_{d}) rate. Equilibrium affinity constant, K, is the ratio of Kₐ/K_{d}. Association (Kₐ) and dissociation (K_{d}) rates can be measured using surface plasmon resonance (SPR) (Rich and Myszka, Curr. Opin. Biotechnol. 11:54 (2000); Englebienne, Analyst. 123:1599 (1998)). Instrumentation and methods for real time detection and monitoring of binding rates are known and are commercially available (BiaCore 2000, Biacore AB, Upsala, Sweden; and Malmqvist, Biochem. Soc. Trans. 27:335 (1999)).

Additional specific non-limiting antibodies and functional fragments have binding affinity for a cell or antigen to which BARB3 antibody, as represented by DSMZ Deposit No. DSM ACC2859, or heavy and light chain sequences set forth as SEQ ID NOs:1 and 2, within about K_{d} 10⁻² M to about K_{d} 10⁻¹⁵ M, or within about K_{d} 10⁻⁵ M to about K_{d} 10⁻¹² M. In particular embodiments, binding affinity for is less than 5×10⁻² M, 10⁻² M, 5×10⁻³ M, 10⁻³ M 5×10⁻⁴ M, 10⁻⁴ M 5×10⁻⁵ M, 10⁻⁵ M 5×10⁻⁶ M, 10⁻⁶ M 5×10⁻⁷ M, 10⁻⁷ M 5×10⁻⁸ M, 10⁻⁸ M 5×10⁻⁹ M, 10⁻⁹ M 5×10⁻¹⁰ M, 10⁻¹⁰ M 5×10⁻¹¹ M, 10⁻¹¹ M 5×10⁻¹² M, 10⁻¹² M 5×10⁻¹³ M, 10⁻¹³ M 5×10⁻¹⁴ M, 10⁻¹⁴ M 5×10⁻¹⁵ M, and 10⁻¹⁵ M. In particular embodiments, an antibody or functional fragment has a binding affinity within about K_{d} 10⁻⁵ M to about K_{d} 10⁻¹³ M for binding to a neoplastic, cancer, tumor or metastatic cell. In additional particular embodiments, an antibody or functional fragment has a binding affinity within about K_{d} 10⁻⁵ M to about K_{d} 10⁻¹³ M for binding to an adenocarcinoma cell or a squamous cell carcinoma, such as a stomach adenocarcinoma cell, a lung adenocarcinoma cell, a pancreas adenocarcinoma cell, a colon adenocarcinoma cell, a breast adenocarcinoma cell, or an esophagus squamous cell carcinoma. In further particular embodiments, an antibody or functional fragment has a binding affinity within about K_{d} 10⁻⁵ M to about K_{d} 10⁻¹³ M for binding to a human adenocarcinoma, human squamous cell carcinoma, human carcinoid carcinoma, human invasive ductal carcinoma, or human germ cell carcinoma of any of stomach, lung, colon, pancreas, esophagus, prostate, breast or testis. In still further particular embodiments, an antibody or functional fragment has a binding affinity within about K_{d} 10⁻⁵ M to about K_{d} 10⁻¹³ M for binding to pancreas cancer BXPC-3 cells or stomach cancer 23132/87 cells.

Antibodies and functional fragments that bind to a cell to which BARB3 antibody, as represented by DSMZ Deposit No. DSM ACC2859, or heavy and light chain variable region sequences set forth as SEQ ID NOs:1 and 2, binds, or that compete with BARB3 antibody, as represented by DSMZ Deposit No. DSM ACC2859, or heavy and light chain variable region sequences set forth as SEQ ID NOs:1 and 2, for binding to a cell, can have greater or less relative cell proliferation inhibiting or reducing activity, or greater or less relative cell apoptosis inducing or stimulating activity than BARB3 antibody, as represented by DSMZ Deposit No. DSM ACC2859, or heavy and light chain variable region sequences set forth as SEQ ID NOs:1 and 2. Antibodies and functional fragments of the invention therefore include those that bind to a cell or antigen to which BARB3 antibody, or compete with BARB3 antibody for binding to a cell or antigen, and have greater or less relative cell proliferation inhibiting or reducing activity, or greater or less relative cell apoptosis inducing or stimulating activity than BARB3 antibody, as represented by DSMZ Deposit No. DSM ACC2859, or heavy and light chain variable region sequences set forth as SEQ ID NOs:1 and 2.

Invention antibodies therefore include those that have a sequence distinct from BARB3 antibody, as represented by DSMZ Deposit No. DSM ACC2859, or heavy and light chain variable region sequences set forth as SEQ ID NOs:1 and 2, but that retain one or more activities or functions, at least in part, of BARB3 antibody, as represented by DSMZ Deposit No. DSM ACC2859, or heavy and light chain variable region sequences set forth as SEQ ID NOs:1 and 2. Exemplary activities and functions include, for example, binding to a cell to which BARB3 antibody binds; binding to an antigen to which BARB3 antibody binds; competing with BARB3 antibody for binding to a cell or to an antigen; inhibiting or reducing cell growth or proliferation, or stimulating or inducing cell death, lysis or apoptosis (e.g., a neoplastic, tumor or cancer, or metastasis cell); binding to one or more of an adenocarcinoma cell or a squamous cell carcinoma, such as a stomach adenocarcinoma cell, a lung adenocarcinoma cell, a pancreas adenocarcinoma cell, a colon adenocarcinoma cell, a breast adenocarcinoma cell, or an esophagus squamous cell carcinoma, or a human adenocarcinoma, human squamous cell carcinoma, human carcinoid carcinoma, human invasive ductal carcinoma, or human germ cell carcinoma of any of stomach, lung, colon, pancreas, esophagus, prostate, breast or testis; inhibiting BXPC-3 or 23132/87 cell growth or proliferation, or stimulating or inducing BXPC-3 or 23132/87 cell death, lysis or apoptosis; etc.

Thus, in accordance with the invention there are also provided modified antibodies and functional fragments provided that the modified form retains, at least a part of an activity or function of unmodified or reference antibody, or functional fragment. In one embodiment, an antibody or a functional fragment thereof includes a heavy or a light chain variable region sequence with one or more amino acid additions, deletions or substitutions of BARB3 antibody, as represented by DSMZ Deposit No. DSM ACC2859, or heavy and light chain variable region sequences set forth as SEQ ID NOs:1 and 2, provided said antibody or functional fragment retains at least partial activity or function of intact full length BARB3 antibody, as represented by DSMZ Deposit No. DSM ACC2859, or heavy and light chain variable region sequences set forth as SEQ ID NOs:1 and 2. In one aspect, an antibody or a functional fragment with one or more amino acid additions, deletions or substitutions of BARB3 antibody, as represented by DSMZ Deposit No. DSM ACC2859, or heavy and light chain variable region sequences set forth as SEQ ID NOs:1 and 2, competes for binding to a cell or antigen to which BARB3 antibody, as represented by DSMZ Deposit No. DSM ACC2859, or heavy and light chain variable region sequences set forth as SEQ ID NOs:1 and 2, binds. In another aspect, an antibody or a functional fragment with one or more amino acid deletions, substitutions or additions of BARB3 antibody, as represented by DSMZ Deposit No. DSM ACC2859, or heavy and light chain variable region sequences set forth as SEQ ID NOs:1 and 2, binds to a cell or antigen to which BARB3 antibody binds. In an additional aspect, an antibody or a functional fragment with one or more amino acid deletions, substitutions or additions of BARB3 antibody, as represented by DSMZ Deposit No. DSM ACC2859, or heavy and light chain variable region sequences set forth as SEQ ID NOs:1 and 2, inhibits or reduces proliferation of a cell in which BARB3 antibody inhibits or reduces proliferation. In a further aspect, an antibody or a functional fragment with one or more amino acid deletions, substitutions or additions of BARB3 antibody, as represented by DSMZ Deposit No. DSM ACC2859, or heavy and light chain variable region sequences set forth as SEQ ID NOs:1 and 2, stimulates or induces death, lysis or apoptosis of a cell in which BARB3 antibody stimulates or induces death, lysis or apoptosis. In still further particular aspects, cell growth or proliferation is inhibited, decreased or reduced at least 20%, 30%, 40%, 50%, 60%, 75%, or more relative to a control (untreated) cell, or any numerical value or range within or encompassing such percent values. In yet further particular aspects, cell death, lysis or apoptosis is at least 20%, 30%, 40%, 50%, 60%, 75%, or more relative to a control (untreated) cell, or any numerical value or range within or encompassing such percent values.

As used herein, the term “modify” and grammatical variations thereof, means that the composition deviates from a reference composition. Such modified proteins, nucleic acids and other compositions may have greater or less activity than or a distinct function from a reference unmodified protein, nucleic acid, or composition.

Modifications, which include substitutions, additions and deletions, can also be referred to as “variants.” Specific non-limiting examples of amino acid variants include BARB3 antibody fragments and subsequences. Exemplary BARB3 antibody subsequences and fragments include a portion of the BARB3 antibody, as represented by DSMZ Deposit No. DSM ACC2859, or heavy and light chain variable region sequences set forth as SEQ ID NOs:1 and 2, that at least partially competes with BARB3 antibody for binding to a cell or antigen, or that retains at least partial binding activity to a cell or antigen to which BARB3 antibody, as represented by DSMZ Deposit No. DSM ACC2859, or heavy and light chain variable region sequences set forth as SEQ ID NOs:1 and 2, binds, or that retains an ability to inhibit or reduce proliferation of a cell in which BARB3 antibody inhibits or reduces proliferation, or that retains an ability to stimulate or induce death, lysis or apoptosis of a cell in which BARB3 antibody stimulates or induces death, lysis or apoptosis.

As used herein, the term “fragment” or “subsequence” means a portion of the full length molecule. Thus, a fragment or subsequence of an antibody has one or more less amino acids than a full length intact reference antibody (e.g. one or more internal or terminal amino acid deletions from either amino or carboxy-termini of heavy or light chain variable or constant regions). A nucleic acid fragment has at least one less nucleotide than a full length comparison nucleic acid sequence. Fragments therefore can be any length up to the full length native molecule.

The terms “functional fragment” and “functional subsequence” when referring to an antibody refers to a portion of an antibody with a function or activity. For example, a functional fragment can retain one or more partial functions or activities as an intact reference antibody, e.g. a function or activity of BARB3 antibody, as represented by DSMZ Deposit No. DSM ACC2859, or heavy and light chain sequences set forth as SEQ ID NOs:1 and 2. For example, a BARB3 antibody subsequence that competes for binding of full length intact BARB3 antibody, as represented by DSMZ Deposit No. DSM ACC2859; or heavy and light chain sequences set forth as SEQ ID NOs:1 and 2, to a cell or to an antigen, or that binds to a cell or antigen to which full length intact BARB3 antibody binds is considered a functional subsequence.

Antibody fragments, including single-chain antibodies, can include all or a portion of heavy or light chain variable region(s) (e.g., one or more CDRs, such as CDR1, CDR2 or CDR3) alone or in combination with all or a portion of one or more of the following: hinge region, CH1, CH2, and CH3 domains. Also included are antigen-binding subsequences of any combination of heavy or light chain variable region(s) (e.g., one or more CDRs, such as CDR1, CDR2 or CDR3) with a hinge region, CH₁, CH2, and CH3 domains.

Exemplary antibody subsequences and fragments of the invention include Fab, Fab′, F(ab′)₂, Fv, Fd, single-chain Fv (scFv), disulfide-linked Fvs (sdFv), V_{L} and V_{H} domain fragments, trispecific (Fab₃), bispecific (Fab₂), diabody ((V_{L}-V_{H})₂ or (V_{H}-V_{L})₂), triabody (trivalent), tetrabody (tetravalent), minibody ((scFᵥ-C_{H}3)₂), bispecific single-chain Fv (Bis-scFv), IgGdeltaCH2, scFv-Fc and (scFv)₂-Fc. Such subsequences and fragments can have binding affinity as the full length antibody, the binding specificity as the full length antibody, or one or more activities or functions of as a full length antibody, e.g., a function or activity of BARB3 antibody, as represented by DSMZ Deposit No. DSM ACC2859, or heavy and light chain sequences set forth as SEQ ID NOs:1 and 2.

Antibody subsequences and fragments can be combined. For example, a V_{L} or V_{H} subsequences can be joined by a linker sequence thereby forming a V_{L}-V_{H} chimera. In particular, a heavy chain variable sequence of BARB3 antibody as represented by DSMZ Deposit No. DSM ACC2859, or heavy chain variable sequence set forth as SEQ ID NO:1, can be combined with a light chain variable sequence of BARB3 antibody as represented by DSMZ Deposit No. DSM ACC2859, or light chain variable sequence set forth as SEQ ID NO:2. The invention therefore provides: 1) heavy chain variable sequence of BARB3 antibody, as represented by DSMZ Deposit No. DSM ACC2859, or heavy chain variable sequence set forth as SEQ ID NO:1; and 2) light chain variable sequence of BARB3 antibody, as represented by DSMZ Deposit No. DSM ACC2859, or light chain variable sequence set forth as SEQ ID NO:2 alone and in combination with each other. A combination of single-chain Fvs (scFv) subsequences can be joined by a linker sequence thereby forming a scFv-scFv chimera. Antibody subsequences and fragments include single-chain antibodies or variable region(s) alone or in combination with all or a portion of other subsequences.

Modified proteins further include amino acid substitutions. Substitutions can be conservative or non-conservative and may be in a constant or variable (e.g., hypervariable, such as CDR or FR) region of BARB3 antibody, as represented by DSMZ Deposit No. DSM ACC2859, or heavy and light chain sequences set forth as SEQ ID NOs:1 and 2. In particular embodiments, a modified BARB3 antibody, as represented by DSMZ Deposit No. DSM ACC2859, or heavy and light chain sequences set forth as SEQ ID NOs:1 and 2, has one or a few conservative or non-conservative amino acid substitutions.

Antibody structural determinants that contribute to antigen binding, such as complemetarity determining regions (CDR, of which there are three in each heavy and light chain sequence, conveniently denoted as HC-CDR1, HC-CDR2 and HC-CDR3; and LC-CDR1, LC-CDR2 and LC-CDR3) within hypervariable regions are known to the skilled artisan. The location of additional regions, such as D- and J-regions are also known to the skilled artisan. Antibodies and subsequences thereof in which one or more CDR sequences have sufficient sequence identity to BARB3 antibody, as represented by DSMZ Deposit No. DSM ACC2859, or heavy and light chain sequences set forth as SEQ ID NOs:1 and 2, so as to retain at least partial function or activity of BARB3 antibody, as represented by DSMZ Deposit No. DSM ACC2859, or heavy and light chain sequences set forth as SEQ ID NOs:1 and 2, e.g., cell or antigen binding, binding affinity (e.g., K_{d}), cell proliferation inhibition, or stimulating or inducing cell apoptosis, etc.

Accordingly, amino acid substitutions in constant or variable regions of BARB3 antibody, as represented by DSMZ Deposit No. DSM ACC2859, or heavy and light chain sequences set forth as SEQ ID NOs:1 and 2, are likely to be tolerated. One or a few substitutions in a variable region outside of a CDR of BARB3 antibody, as represented by DSMZ Deposit No. DSM ACC2859, or heavy and light chain sequences set forth as SEQ ID NOs:1 and 2, is also likely to be tolerated at least to the extent that at least partial cell or antigen binding activity is retained, or partial cell proliferation inhibiting or apoptosis stimulating or inducing activity is retained. One or a few conservative substitutions in a CDR of BARB3 antibody, as represented by DSMZ Deposit No. DSM ACC2859, or heavy and light chain sequences set forth as SEQ ID NOs:1 and 2, is also likely to be tolerated at least to the extent that at least partial cell or antigen binding activity is retained (i.e., cell or antigen binding is not destroyed), or partial cell proliferation inhibiting or apoptosis stimulating or inducing activity is retained. Non-conservative substitution of many amino acids in hypervariable regions (e.g., CDRs) of BARB3 antibody, as represented by DSMZ Deposit No. DSM ACC2859, or heavy and light chain sequences set forth as SEQ ID NOs:1 and 2, is likely to affect one or more of cell or antigen binding activity, binding affinity (e.g., K_{d}), or antibody function or activity, such as cell proliferation inhibition, stimulating or inducing cell apoptosis, etc.

A “conservative substitution” is the replacement of one amino acid by a biologically, chemically or structurally similar residue. Biologically similar means that the substitution does not destroy a biological activity, e.g., cell binding or cell proliferation inhibiting or apoptosis inducing or stimulating activity. Structurally similar means that the amino acids have side chains with similar length, such as alanine, glycine and serine, or a similar size. Chemical similarity means that the residues have the same charge or are both hydrophilic or hydrophobic. Particular examples include the substitution of one hydrophobic residue, such as isoleucine, valine, leucine or methionine for another, or the substitution of one polar residue for another, such as the substitution of arginine for lysine, glutamic for aspartic acids, or glutamine for asparagine, serine for threonine, and the like.

In particular embodiments, a heavy or light chain hypervariable region sequence or a region therein, such as a CDR (CDR1, CDR2 or CDR3) or FR will have 1-10, 1-5, 1-3 or fewer (e.g., 1 or 2) amino acid substitutions. In an additional embodiment, an amino acid substitution within a heavy or light chain hypervariable region sequence is not within more than one CDR. In an additional embodiment, a substitution within a heavy or light chain hypervariable region sequence is not within a CDR. In another embodiment, a substitution within a hypervariable region sequence is not within an FR.

The effect of a given modification can be readily assayed in order to identify antibodies and functional fragments retaining at least a part of the cell or antigen binding activity, affinity or antibody function or activity of unmodified antibody, e.g., BARB3 antibody, as represented by DSMZ Deposit No. DSM ACC2859, or heavy and light chain sequences set forth as SEQ ID NOs:1 and 2. For example, an amino acid substitution in a variable region (e.g., within or outside of CDR1, CDR2 or CDR3 of BARB3 antibody, as represented by DSMZ Deposit No. DSM ACC2859, or heavy and light chain sequences set forth as SEQ ID NOs:1 and 2) can be assayed for cell or antigen binding, cell proliferation inhibiting or reducing activity, inducing or stimulating cell death, lysis or apoptosis, etc.

Regional mutability analysis can be used to predict the effect of particular substitutions in complementarity determining regions (CDR) and framework regions (FR) (Shapiro et al., J Immunol. 163:259 (1999)). In brief, sequence comparison indicates a hierarchy of mutability among di- and trinucleotide sequences located within Ig intronic DNA, which predicts regions that are more or less mutable. Quantitative structure-activity relationship (QSAR) can be used to identify the nature of the antibody recognition domain and, therefore, amino acids that participate in ligand binding. Predictive models based upon OSAR can in turn be used to predict the effect of substitutions (mutations). For example, the effect of mutations on the association and dissociation rate of an antibody interacting with its antigen has been used to construct quantitative predictive models for both kinetic (Kₐ and K_{d}) constants, which in turn is used to predict the effect of other mutations on the antibody (De Genst et al., J Biol. Chem. 277:29897 (2002)). The skilled artisan can therefore use such analysis to identify amino acid substitutions of antibodies and functional fragments that are likely to result in an antibody or functional fragment that retains at least partial activity or function of non-substituted antibody or functional fragment.

Amino acid substitutions may be with the same amino acid, except that a naturally occurring L-amino acid is substituted with a D-form amino acid. Modifications therefore include one or more D-amino acids substituted for L-amino acids, or mixtures of D-amino acids substituted for L-amino acids. Modifications also include structural and functional analogues, for example, peptidomimetics having synthetic or non-natural amino acids or amino acid analogues and derivatized forms.

Modified forms further include derivatized sequences, for example, amino acids in which free amino groups form amine hydrochlorides, p-toluene sulfonyl groups, carbobenzoxy groups; the free carboxy groups from salts, methyl and ethyl esters; free hydroxyl groups that form O-acyl or O-alkyl derivatives, as well as naturally occurring amino acid derivatives, for example, 4-hydroxyproline, for proline, 5-hydroxylysine for lysine, homoserine for serine, ornithine for lysine, etc. Modifications can be produced using methods known in the art (e.g., PCR based site-directed, deletion and insertion mutagenesis, chemical modification and mutagenesis, cross-linking, etc.).

Modified forms include additions and insertions. For example, an addition can be the covalent or non-covalent attachment of any type of molecule to a protein (e.g., antibody), nucleic acid or other composition. Typically additions and insertions confer a distinct function or activity.

Additions and insertions include fusion (chimeric) polypeptide or nucleic acid sequences, which is a sequence having one or more molecules not normally present in a reference native (wild type) sequence covalently attached to the sequence. A particular example is an amino acid sequence of another protein (e.g., antibody) to produce a multifunctional protein (e.g., multispecific antibody).

In accordance with the invention, there are provided antibodies, nucleic acids, and other compositions that include a heterologous domain. Thus, a heterologous domain can consist of any of a variety of different types of small or large functional moieties. Such moieties include nucleic acid, peptide, carbohydrate, lipid or small organic compounds, such as a drug (e.g., a cell proliferative agent), metals (gold, silver), etc. A heterologous domains can be an amino acid addition or insertion.

Particular non-limiting examples of heterologous domains include, for example, tags, detectable labels and cytotoxic agents. Specific examples of tags and detectable labels include enzymes (horseradish peroxidase, urease, catalase, alkaline phosphatase, beta-galactosidase, chloramphenicol transferase); enzyme substrates; ligands (e.g., biotin); receptors (avidin); radionuclides (e.g., C¹⁴, S³⁵, P³², P³³, H³, I¹²⁵, I¹³¹, gallium-67 and 68, scantium-47, indium-111, radium-223); T7-, His-, myc-, HA- and FLAG-tags; electron-dense reagents; energy transfer molecules; paramagnetic labels; fluorophores (fluorescein, rhodamine, phycoerythrin); chromophores; chemi-luminescent (imidazole, luciferase); and bio-luminescent agents. Specific examples of cytotoxic agents include diphtheria, toxin, cholera toxin and ricin.

Additional examples of heterologous domains include, for example, anti-cell proliferative agents (e.g., anti-neoplastic, anti-tumor or anti-cancer, or anti-metastasis agents). Specific non-limiting examples of anti-cell proliferative agents are disclosed herein and known in the art.

Linker sequences may be inserted between the protein (e.g., antibody), nucleic acid, or other composition and the addition or insertion (e.g., heterologous domain) so that the two entities maintain, at least in part, a distinct function or activity. Linker sequences may have one or more properties that include a flexible structure, an inability to form an ordered secondary structure or a hydrophobic or charged character which could promote or interact with either domain. Amino acids typically found in flexible protein regions include Gly, Asn and Ser. Other near neutral amino acids, such as Thr and Ala, may also be used in the linker sequence. The length of the linker sequence may vary (see, e.g., U.S. Pat. No. 6,087,329). Linkers further include chemical cross-linking and conjugating agents, such as sulfo-succinimidyl derivatives (sulfo-SMCC, sulfo-SMPB), disuccinimidyl suberate (DSS), disuccinimidyl glutarate (DSG) and disuccinimidyl tartrate (DST).

Further examples of additions include glycosylation, fatty acids, lipids, acetylation, phosphorylation, amidation, formylation, ubiquitinatation, and derivatization by protecting/blocking groups and any of numerous chemical modifications. Other permutations and possibilities will be readily apparent to those of ordinary skill in the art, and are considered to be within the scope of the invention.

The term “isolated” used as a modifier of a composition means that the composition is made by the hand of man or is separated from one or more other components in their naturally occurring in vivo environment. Generally, compositions so separated are substantially free of one or more materials with which they normally associate with in nature, for example, one or more protein, nucleic acid, lipid, carbohydrate, cell membrane. Thus, an isolated composition is substantially separated from other biological components in the cell of the organism in which the composition naturally occurs, or from the artificial medium in which it is produced (e.g., synthetically or through cell culture). For example, an isolated polypeptide is substantially separated from other polypeptides and nucleic acid and does not include a library of polypeptides or polynucleotides present among millions of polypeptide or nucleic acid sequences, such as a polypeptide, genomic or cDNA library, for example. An isolated nucleic acid is substantially separated from other polypeptides and nucleic acid and does not include a library of polypeptides or polynucleotides present among millions of polypeptide or nucleic acid sequences, such as a polypeptide, genomic or cDNA library, for example. The term “isolated” does not exclude alternative physical forms of the composition, for example, an isolated protein could include protein multimers, post-translational modifications (e.g., glycosylation, phosphorylation) or derivatized forms.

The term “purified” used as a modifier of a composition refers to a composition free of most or all of the materials with which it typically associates with in nature. Thus, a protein separated from cells is considered to be substantially purified when separated from cellular components by standard methods while a chemically synthesized nucleic acid sequence is considered to be substantially purified when separated from its chemical precursors. Purified therefore does not require absolute purity. Furthermore, a “purified” composition can be combined with one or more other molecules. Thus, the term “purified” does not exclude combinations of compositions.

“Purified” proteins and nucleic acid include proteins and nucleic acids produced by standard purification methods. The term also includes proteins and nucleic acids produced by recombinant expression in a host cell as well as chemical synthesis. “Purified” can also refer to a composition in which the level of contaminants is below a level that is acceptable to a regulatory agency for administration to a human or non-human animal, for example, the Food and Drug administration (FDA).

Substantial purity can be at least about 60% or more of the molecule by mass. Purity can also be about 70% or 80% or more, and can be greater, for example, 90% or more. Purity can be less, for example, in a pharmaceutical carrier the amount of a molecule by weight % can be less than 60% but the relative proportion of the molecule compared to other components with which it is normally associated with will be greater. Purity can be determined by any appropriate method, including, for example, UV spectroscopy, chromatography (e.g., HPLC, gas phase), gel electrophoresis (e.g., silver or coomassie staining) and sequence analysis (peptide and nucleic acid).

Methods of producing polyclonal and monoclonal antibodies are known in the art. For example, BARB3 antigen or an immunogenic fragment thereof, optionally conjugated to a carrier such as keyhole limpet hemocyanin (KLH) or ovalbumin (e.g., BSA), or mixed with an adjuvant such as Freund's complete or incomplete adjuvant, and used to immunize an animal. Using conventional hybridoma technology, splenocytes from immunized animals that respond to BARB3 antigen can be isolated and fused with myeloma cells. Monoclonal antibodies produced by the hybridomas can be screened for reactivity with BARB3 antigen.

Antibodies that compete with BARB3 antibody, as represented by DSMZ Deposit No. DSM ACC2859, or heavy and light chain variable region sequences set forth as SEQ ID NOs:1 and 2, for binding to a cell or antigen can be screened and identified using a conventional competition binding assays. Screened antibodies are selected based upon an ability to compete with BARB3 antibody, as represented by DSMZ Deposit No. DSM ACC2859, or heavy and light chain variable region sequences set forth as SEQ ID NOs:1 and 2, for binding to a cell or antigen. The ability of an antibody to compete with BARB3 antibody for binding to a cell or antigen, or to inhibit, prevent or block binding of BARB3 antibody to a cell or antigen, can be determined by various assays know in the art, including enzyme linked immunosorbent assay (ELISA).

Proteins and antibodies, subsequences and fragments thereof, as well as other modified sequences can be produced by genetic methodology. Such techniques include expression of all or a part of the gene encoding the protein or antibody into a host cell such as Cos cells or E. coli. Such host cells can express full length or a fragment, for example, an scFv (see, e.g., Whitlow et al., In: Methods: A Companion to Methods in Enzymology 2:97 (1991), Bird et al., Science 242:423 (1988); and U.S. Pat. No. 4,946,778). Antibodies and functional fragments, and nucleic acid sequences can also be produced by chemical synthesis using methods known to the skilled artisan, for example, an automated peptide synthesis apparatus (see, e.g., Applied Biosystems, Foster City, Calif.). Antibodies and functional fragments can be screened or selected using various assays know in the art, such as enzyme linked immunosorbent assay (ELISA), phage display, protein-mRNA link via ribosome and mRNA display, display on yeast, bacteria, mammalian cells or retroviruses, microbead via in vitro compartmentalization, protein-DNA display, growth selection via yeast 2-hybrid, protein fragment complementation (Hoogenboom, R., Nature Biotechnol. 23:1105 (2005)).

Cells or antigen suitable for generating antibodies can be produced by any of a variety of standard protein purification or recombinant expression techniques known in the art. For example, BARB3 antigen is present on cells, such as BXPC-3 cells (ATCC Deposit No. CRL-1687; P.O. Box 1549 Manassas, Va., 20108, USA) or 23132/87 cells (DSMZ Deposit No. ACC 201; Deutsche Sammlung von Mikroorganismen und Zellkulturen GmbH (German Collection of Microorganisms and Cell Cultures), Inhoffenstrase 7 B 38124 Braunschweig, Germany). Accordingly, whole cells, or preparations, cell extracts or fractions of such cells can be used to immunize animals in order to produce antibodies that compete with BARB3 antibody, as represented by DSMZ Deposit No. DSM ACC2859, or heavy and light chain sequences set forth as SEQ ID NOs:1 and 2, for binding of to a cell or antigen, or that bind to a cell or antigen to which BARB3 antibody, as represented by DSMZ Deposit No. DSM ACC2859, or heavy and light chain sequences set forth as SEQ ID NOs:1 and 2, binds, for example.

Animals that may be immunized include mice, rats, rabbits, goats, sheep, cows or steer, guinea pigs or primates. Initial and any optional subsequent immunization may be through intravenous, intraperitoneal, intramuscular, or subcutaneous routes. Subsequent immunizations may be at the same or at different concentrations of BARB3 antigen preparation, and may be at regular or irregular intervals.

Animals include those genetically modified to include human IgG gene loci, which can therefore be used to produce human antibodies. Transgenic animals with one or more human immunoglobulin genes that do not express endogenous immunoglobulins are described, for example in, U.S. Pat. No. 5,939,598. Additional methods for producing human polyclonal antibodies and human monoclonal antibodies are described (see, e.g., Kuroiwa et al., Nat. Biotechnol. 20:889 (2002); WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; and 5,939,598). An overview of the technology for producing human antibodies is described in Lonberg and Huszar (Int. Rev. Immunol. 13:65 (1995)).

Antibodies can also be generated using other techniques including hybridoma, recombinant, and phage display technologies, or a combination thereof (see U.S. Pat. Nos. 4,902,614, 4,543,439, and 4,411,993; see, also Monoclonal Antibodies, Hybridomas: A New Dimension in Biological Analyses, Plenum Press, Kennett, McKearn, and Bechtol (eds.), 1980, and Harlow et al., Antibodies: A Laboratory Manual, Cold Spring Harbor Laboratory Press, 2nd ed. 1988).

Antibody subsequences and fragments can be prepared by proteolytic hydrolysis of the antibody, for example, by pepsin or papain digestion of whole antibodies. Antibody subsequences and fragments produced by enzymatic cleavage with pepsin provide a 5S fragment denoted F(ab′)₂. This fragment can be further cleaved using a thiol reducing agent to produce 3.5S Fab′ monovalent fragments. Alternatively, an enzymatic cleavage using pepsin produces two monovalent Fab′ fragments and the Fe fragment directly (see, e.g., U.S. Pat. Nos. 4,036,945 and 4,331,647; and Edelman et al., Methods Enymol. 1:422 (1967)). Single-chain Fvs and antibodies can be produced as described in U.S. Pat. Nos. 4,946,778 and 5,258,498; Huston et al., Methods Enzymol. 203:46 (1991); Shu et al., Proc. Natl. Acad. Sci. USA 90:7995 (1993); and Skerra et al., Science 240:1038 (1988). Other methods of cleaving antibodies, such as separation of heavy chains to form monovalent light-heavy chain fragments, further cleavage of fragments, or other enzymatic or chemical may also be used.

Modified antibodies and functional fragments having altered characteristics, such as increased binding affinity, can be produced using methods known to the skilled artisan art. For example, affinity maturation techniques can be used to improve antibody binding affinity (US 2004/0162413 A1; U.S. Pat. Nos. 6,656,467, 6,531,580, 6,590,079 and 5,955,358; Fiedler et al., Protein Eng. 15:931 (2002); Pancook et al., Hybrid. Hybridomics 20:383 (2001); Daugherty et al., Protein Eng. 11:825 (1998); Wu et al., Proc. Nat'l Acad. Sci. USA 95:6037 (1998); and Osbourn et al., Immunotechnology 2:181 (1996)).

Antibodies can be humanized using a variety of techniques known in the art including, for example, CDR-grafting (EP 239,400; WO91/09967; U.S. Pat. Nos. 5,225,539; 5,530,101; and 5,585,089), veneering or resurfacing (EP 592,106; EP 519,596; Padlan, Molecular Immunol. 28:489 (1991); Studnicka et al., Protein Engineering 7:805 (1994); Roguska. et al., Proc. Nat'l. Acad. Sci. USA 91:969 (1994)), and chain shuffling (U.S. Pat. No. 5,565,332). Human consensus sequences (Padlan, Mol. Immunol. 31:169 (1994); and Padlan, Mol. Immunol. 28:489 (1991)) have previously used to produce humanized antibodies (Carter et al., Proc. Natl. Acad. Sci. USA 89:4285 (1992); and Presta et al., J. Immunol. 151:2623 (1993)).

Methods for producing chimeric antibodies are known in the art (e.g., Morrison, Science 229:1202 (1985); Oi et al, BioTechniques 4:214 (1986); Gillies et al., J. Immunol. Methods 125:191 (1989); and U.S. Pat. Nos. 5,807,715; 4,816,567; and 4,816,397). Chimeric antibodies in which a variable domain from an antibody of one species is substituted for the variable domain of another species are described, for example, in Munro, Nature 312:597 (1984); Neuberger et al., Nature 312:604 (1984); Sharon et al., Nature 309:364 (1984); Morrison et al., Proc. Nat'l. Acad. Sci. USA 81:6851 (1984); Boulianne et al., Nature 312:643 (1984); Capon et al., Nature 337:525 (1989); and Traunecker et al., Nature 339:68 (1989).

Suitable techniques that additionally may be employed in antibody methods include affinity purification, non-denaturing gel purification, HPLC or RP-HPLC, size exclusion, purification on protein A column, or any combination of these techniques. The antibody isotype can be determined using an ELISA assay, for example, a human Ig can be identified using mouse Ig-absorbed anti-human Ig.

In accordance with the invention, further provided are methods of producing antibodies and functional fragments. In one embodiment, a method includes administering a BARB3 antigen, or cell expressing a BARB3 antigen, to an animal, screening the animal for expression of an antibody that binds to the BARB3 antigen or cell expressing a BARB3 antigen, selecting an animal that produces an antibody that binds to BARB3 antigen or cell expressing a BARB3 antigen, and isolating the antibody from the selected animal. In another embodiment, a method includes administering BARB3 antigen or cell expressing a BARB3 antigen to an animal capable of expressing a human immunoglobulin; isolating spleen cells from an animal that produces antibody that binds to the BARB3 antigen or cell expressing a BARB3 antigen, fusing the spleen cells with a myeloma cell to produce a hybridoma, and screening the hybridoma for expression of an antibody that binds to BARB3 antigen or cell expressing a BARB3 antigen.

In accordance with the invention, there are provided host cells that express antibodies and functional fragments of the antibodies as set forth herein. In particular embodiments, host cells are purified or isolated, and optionally have not been transformed with a nucleic acid that encodes the expressed antibody or functional fragment. In additional embodiments, a host cell expresses an antibody or functional fragment that includes a heavy or light chain sequence with 50%, 60%, 70%, 75%, 80%, 85%, 90%, 95%, 96%, 97%, 98%, or more sequence identity to BARB3 antibody, as represented by DSMZ Deposit No. DSM ACC2859, or heavy and light chain variable region sequences set forth as SEQ ID NOs:1 and 2. In further embodiments, a host cell expresses a heavy or light chain sequence with at least 80-85%, 85-90%, 90-95%, 95-100% identity to one or more CDRs in heavy chain variable region sequence or light chain variable region sequence BARB3 antibody, as represented by DSMZ Deposit No. DSM ACC2859, or heavy and light chain variable region sequences set forth as SEQ ID NOs:1 and 2.

In accordance with the invention, there are provided isolated and purified nucleic acids. Nucleic acids of the invention include, among other things, nucleic acid sequences 1) encoding antibodies and functional fragments that are structurally or functionally related to BARB3 antibody, as represented by DSMZ Deposit No. DSM ACC2859; or heavy and light chain sequences set forth as SEQ ID NOs:1 and 2; 2) encode SEQ ID NOs:1 or 2, or antibodies and functional fragments that include all or a portion of a sequence of SEQ ID NOs:1 or 2 (e.g., one or more CDRs); 3) that exhibit a degree of complementarity or identity with nucleic acid sequences encoding antibodies and functional fragments with sequence identity to BARB3 antibody, as represented by DSMZ Deposit No. DSM ACC2859 or heavy and light chain sequences set forth as SEQ ID NOs:1 and 2; and 4) that hybridize to sequences encoding antibodies and functional fragments that have sequence identity to BARB3 antibody, as represented by DSMZ Deposit No. DSM ACC2859, or heavy and light chain sequences set forth as SEQ ID NOs:1 and 2.

In particular embodiments, a nucleic acid sequence encodes a heavy or light chain sequence of BARB3 antibody, as represented by DSMZ Deposit No. DSM ACC2859, or heavy and light chain sequences set forth as SEQ ID NOs:1 and 2, or a functional fragment thereof. In another embodiment, a nucleic acid sequence is 75-100% complementary or identical to a nucleic acid sequence that encodes SEQ ID NO:1. In a further embodiment, a nucleic acid sequence is 75-100% complementary or identical to a nucleic acid sequence that encodes SEQ ID NO:2.

Proteins, such as antibodies that include amino acid substitutions, additions or deletions can be encoded by a nucleic acid. Consequently, nucleic acid sequences encoding proteins that include amino acid substitutions, additions or deletions are also provided.

The terms “nucleic acid” and “polynucleotide” and the like refer to at least two or more ribo- or deoxy-ribonucleic acid base pairs (nucleotides) that are linked through a phosphoester bond or equivalent. Nucleic acids include polynucleotides and polynucleosides. Nucleic acids include single, double or triplex, circular or linear, molecules. Exemplary nucleic acids include but are not limited to: RNA, DNA, cDNA, genomic nucleic acid, naturally occurring and non naturally occurring nucleic acid, e.g., synthetic nucleic acid.

Nucleic acids can be of various lengths. Nucleic acid lengths typically range from about 20 nucleotides to 20 Kb, or any numerical value or range within or encompassing such lengths, 10 nucleotides to 10 Kb, 1 to 5 Kb or less, 1000 to about 500 nucleotides or less in length. Nucleic acids can also be shorter, for example, 100 to about 500 nucleotides, or from about 12 to 25, 25 to 50, 50 to 100, 100 to 250, or about 250 to 500 nucleotides in length, or any numerical value or range or value within or encompassing such lengths. In particular embodiments, a nucleic acid sequence has a length from about 10-20, 20-30, 30-50, 50-100, 100-150, 150-200, 200-250, 250-300, 300-400, 400-500, 500-1000, 1000-2000, nucleotides, or any numerical value or range within or encompassing such lengths. Shorter polynucleotides are commonly referred to as “oligonucleotides” or “probes” of single- or double-stranded DNA. However, there is no upper limit to the length of such oligonucleotides.

Polynucleotides include L- or D-forms and mixtures thereof, which additionally may be modified to be resistant to degradation when administered to a subject. Particular examples include 5′ and 3′ linkages resistant to endonucleases and exonucleases present in various tissues or fluids of a subject.

In accordance with the invention there are provided nucleic acid sequences that hybridize to a nucleic acid that encodes all or a fragment of a BARB3 antibody, as represented by DSMZ Deposit No. DSM ACC2859, or heavy and light chain sequences set forth as SEQ ID NOs:1 and 2. In one embodiment, a nucleic acid sequence specifically hybridizes to a nucleic acid encoding SEQ ID NO:1 or a portion thereof. In another embodiment, a nucleic acid sequence specifically hybridizes to a nucleic acid encoding SEQ ID NO:2 or a portion thereof. In a further embodiment, a nucleic acid sequence is at least 75-100% complementary or homologous to a nucleic acid sequence that encodes all or a subsequence or fragment of BARB3 antibody, as represented by DSMZ Deposit No. DSM ACC2859, or heavy and light chain sequences set forth as SEQ ID NOs:1 and 2.

The term “hybridize” and grammatical variations thereof refer to the binding between nucleic acid sequences. Hybridizing sequences will generally have more than about 50% homology (e.g., 50%, 60%, 70%, 80%, 90%, or more identity) to a reference nucleic acid or a sequence complementary to a reference sequence. Hybridizing sequences that are 100% or fully complementary to a reference sequence, for example, to a nucleic acid that encodes an amino acid sequence of a reference sequence, exhibit 100% base pairing with no mismatches. The hybridization region between hybridizing sequences typically is at least about 12-15 nucleotides, 15-20 nucleotides, 20-30 nucleotides, 30-50 nucleotides, 50-100 nucleotides, 100 to 200 nucleotides or more, or any numerical value or range within or encompassing such lengths.

In accordance with the invention, there are further provided antisense polynucleotides, small interfering RNA, and ribozyme nucleic acid. In one embodiment, an antisense polynucleotide, small interfering RNA, or ribozyme nucleic acid specifically hybridizes to a nucleic acid sequence encoding SEQ ID NO:1, 2 or 5, or a portion thereof, and optionally reduces expression of SEQ ID NO:1, 2 or 5. In another embodiment, an antisense polynucleotide, small interfering RNA, or ribozyme nucleic acid is at least 60% or more (e.g., 65%, 70%, 75%, 80%, 85%, 90%, 95%, etc.) complementary or homologous to a nucleic acid sequence that encodes SEQ ID NO:1, 2 or 5, or a subsequence of SEQ ID NOs:1, 2 or 5. Antisense polynucleotides can have a length from about 10-20, 20-30, 30-50, 50-100, 100-150, 150-200, 200-250, 250-300, 300-400, 400-500, 500-1000, 1000-2000 nucleotides, or any numerical value or range within or encompassing such lengths.

As used herein, the term “antisense” refers to a polynucleotide or peptide nucleic acid capable of binding to a specific DNA or RNA sequence. Antisense includes single, double, triple or greater stranded RNA and DNA polynucleotides and peptide nucleic acids (PNAs) that bind RNA transcript or DNA. Particular examples include RNA and DNA antisense that binds to sense RNA. For example, a single stranded nucleic acid can target a protein transcript that participates in metabolism, catabolism, removal or degradation of glycogen from a cell (e.g., mRNA). Antisense molecules are typically 95-100% complementary to the sense strand but can be “partially” complementary, in which only some of the nucleotides bind to the sense molecule (less than 100% complementary, e.g., 95%, 90%, 80%, 70% and sometimes less), or any numerical value or range within or encompassing such percent values.

Triplex forming antisense can bind to double strand DNA thereby inhibiting transcription of the gene. Oligonucleotides derived from the transcription initiation site of the gene, e.g., between positions −10 and +10 from the start site, are one particular example.

Short interfering RNA (referred to as siRNA or RNAi) for inhibiting gene expression is known in the art (see, e.g., Kennerdell et al., Cell 95:1017 (1998); Fire et al., Nature, 391:806 (1998); WO 02/44321; WO 01/68836; WO 00/44895, WO 99/32619, WO 01/75164, WO 01/92513, WO 01/29058, WO 01/89304, WO 02/16620; and WO 02/29858). RNAi silencing can be induced by a nucleic acid encoding an RNA that forms a “hairpin” structure or by expressing RNA from each end of an encoding nucleic acid, making two RNA molecules that hybridize.

Ribozymes, which are enzymatic RNA molecules that catalyze the specific cleavage of RNA can be used to inhibit expression of the encoded protein. Ribozymes form sequence-specific hybrids with complementary target RNA, which is then cleaved. Specific examples include engineered hammerhead motif ribozyme molecules that can specifically and efficiently catalyze endonucleolytic cleavage of sequences encoding a protein that participates in metabolism, catabolism, removal or degradation of glycogen, for example.

Antisense, ribozymes, RNAi and triplex forming nucleic acid are referred to collectively herein as “inhibitory nucleic acid” or “inhibitory polynucleotides.” Such inhibitory nucleic acid or polynucleotides can inhibit or reduce expression of the sequence to which it binds or targets, and consequently, encoded protein as appropriate.

Inhibitory polynucleotides do not require expression control elements in order to function in vivo. Inhibitory polynucleotides can be absorbed by the cell or enter the cell via passive diffusion. Inhibitory polynucleotides can optionally be introduced into a cell using a vector. Inhibitory polynucleotides may be encoded by a nucleic acid so that it is transcribed. Furthermore, a nucleic acid encoding an inhibitory polynucleotide may be operatively linked to an expression control element for sustained or increased expression of the encoded antisense in cells or in vivo. Inhibitory nucleic acid can be designed based upon protein and nucleic acid sequences disclosed herein or available in the database.

Nucleic acid sequences further include nucleotide and nucleoside substitutions, additions and deletions, as well as derivatized forms and fusion/chimeric sequences (e.g., encoding recombinant polypeptide). For example, due to the degeneracy of the genetic code, nucleic acids include sequences and subsequences degenerate with respect to nucleic acids that encode, modified forms and variants thereof. Other examples are nucleic acids complementary to a sequence that encodes

Nucleic acid deletions (subsequences and fragments) can have from about 10 to 25, 25 to 50 or 50 to 100 nucleotides. Such nucleic acids are useful for expressing polypeptide subsequences, for genetic manipulation (as primers and templates for PCR amplification), and as probes to detect the presence or an amount of a sequence encoding a protein (e.g., via hybridization), in a cell, culture medium, biological sample (e.g., tissue, organ, blood or serum), or in a subject.

Nucleic acids can be produced using various standard cloning and chemical synthesis techniques. Techniques include, but are not limited to nucleic acid amplification, e.g., polymerase chain reaction (PCR), with genomic DNA or cDNA targets using primers (e.g., a degenerate primer mixture) capable of annealing to antibody encoding sequence. Nucleic acids can also be produced by chemical synthesis (e.g., solid phase phosphoramidite synthesis) or transcription from a gene. The sequences produced can then be translated in vitro, or cloned into a plasmid and propagated and then expressed in a cell (e.g., a host cell such as yeast or bacteria, a eukaryote such as an animal or mammalian cell or in a plant).

In accordance with the invention, there are further provided vectors that comprise nucleic acid sequences of the invention. In one embodiment, a vector includes a nucleic acid sequence encoding an antibody or functional fragment as set forth herein. In another embodiment, a vector includes a nucleic acid sequence encoding

Vectors include viral, prokaryotic (bacterial) and eukaryotic (plant, fungal, mammalian) vectors. Vectors can be used for expression of nucleic acids in vitro or in vivo. Such vectors, referred to as “expression vectors,” are useful for introducing nucleic acids, including nucleic acids that encode BARB3 antibody, as represented by DSMZ Deposit No. DSM ACC2859, or heavy and light chain variable region sequences set forth as SEQ ID NOs:1 and 2, subsequences and fragments thereof, nucleic acids that encode modified forms or variants of BARB3 antibody, as represented by DSMZ Deposit No. DSM ACC2859, or heavy and light chain variable region sequences set forth as SEQ ID NOs:1 and 2, nucleic acids that encode inhibitory nucleic acid, and expressing the encoded protein or inhibitory nucleic acid (e.g., in solution or in solid phase), in cells or in a subject in vivo.

Vectors can also be used for manipulation of nucleic acids. For genetic manipulation “cloning vectors” can be employed, and to transcribe or translate the inserted nucleic acid.

A vector generally contains an origin of replication for propagation in a cell in vitro or in vivo. Control elements, including expression control elements, present within a vector, can be included to facilitate transcription and translation, as appropriate.

Vectors can include a selection marker. A “selection marker” is a gene that allows for the selection of cells containing the gene. “Positive selection” refers to a process in which cells that contain the selection marker survive upon exposure to the positive selection. Drug resistance is one example of a positive selection marker-cells containing the marker will survive in culture medium containing the selection drug, and cells lacking the marker will die. Selection markers include drug resistance genes such as neo, which confers resistance to G418; hygr, which confers resistance to hygromycin; and puro, which confers resistance to puromycin. Other positive selection marker genes include genes that allow identification or screening of cells containing the marker. These genes include genes for fluorescent proteins (GFP and GFP-like chromophores, luciferase), the lacZ gene, the alkaline phosphatase gene, and surface markers such as CD8, among others. “Negative selection” refers to a process in which cells containing a negative selection marker are killed upon exposure to an appropriate negative selection agent. For example, cells which contain the herpes simplex virus-thymidine kinase (HSV-tk) gene (Wigler et al., Cell 11:223 (1977)) are sensitive to the drug gancyclovir (GANC). Similarly, the gpt gene renders cells sensitive to 6-thioxanthine.

Viral vectors include those based upon retroviral (lentivirus for infecting dividing as well as non-dividing cells), foamy viruses (U.S. Pat. Nos. 5,624,820, 5,693,508, 5,665,577, 6,013,516 and 5,674,703; WO92/05266 and WO92/14829), adenovirus (U.S. Pat. Nos. 5,700,470, 5,731,172 and 5,928,944), adeno-associated virus (AAV) (U.S. Pat. No. 5,604,090), herpes simplex virus vectors (U.S. Pat. No. 5,501,979), cytomegalovirus (CMV) based vectors (U.S. Pat. No. 5,561,063), reovirus, rotavirus genomes, simian virus 40 (SV40) or papilloma virus (Cone et al., Proc. Natl. Acad. Sci. USA 81:6349 (1984); Eukaryotic Viral Vectors, Cold Spring Harbor Laboratory, Gluzman ed., 1982; Sarver et al., Mol. Cell. Biol. 1:486 (1981); U.S. Pat. No. 5,719,054). Adenovirus efficiently infects slowly replicating and/or terminally differentiated cells and can be used to target slowly replicating and/or terminally differentiated cells. Additional viral vectors useful for expression include parvovirus, Norwalk virus, coronaviruses, paramyxo- and rhabdoviruses, togavirus (e.g., sindbis virus and semliki forest virus) and vesicular stomatitis virus (VSV).

A nucleic acid can be expressed when the nucleic acid is operably linked to an expression control element. As used herein, the term “operably linked” refers to a physical or a functional relationship between the elements referred to that permit them to operate in their intended fashion. Thus, an expression control element “operably linked” to a nucleic acid means that the control element modulates nucleic acid transcription and as appropriate, translation of the transcript.

The term “expression control element” refers to nucleic acid that influences expression of an operably linked nucleic acid. Promoters and enhancers are particular non-limiting examples of expression control elements. A “promoter sequence” is a DNA regulatory region capable of initiating transcription of a downstream (3′ direction) sequence. The promoter sequence includes nucleotides that facilitate transcription initiation. Enhancers also regulate gene expression, but can function at a distance from the transcription start site of the gene to which it is operably linked. Enhancers function at either 5′ or 3′ ends of the gene, as well as within the gene (e.g., in introns or coding sequences). Additional expression control elements include leader sequences and fusion partner sequences, internal ribosome binding sites (IPES) elements for the creation of multigene, or polycistronic, messages, splicing signal for introns, maintenance of the correct reading frame of the gene to permit in-frame translation of mRNA, polyadenylation signal to provide proper polyadenylation of the transcript of interest, and stop codons.

Expression control elements include “constitutive” elements in which transcription of an operably linked nucleic acid occurs without the presence of a signal or stimuli. Expression control elements that confer expression in response to a signal or stimuli, which either increase or decrease expression of operably linked nucleic acid, are “regulatable.” A regulatable element that increases expression of operably linked nucleic acid in response to a signal or stimuli is referred to as an “inducible element.” A regulatable element that decreases expression of the operably linked nucleic acid in response to a signal or stimuli is referred to as a “repressible element” (i.e., the signal decreases expression; when the signal is removed or absent, expression is increased).

Expression control elements include elements active in a particular tissue or cell type, referred to as “tissue-specific expression control elements.” Tissue-specific expression control elements are typically more active in specific cell or tissue types because they are recognized by transcriptional activator proteins, or other transcription regulators active in the specific cell or tissue type, as compared to other cell or tissue types.

Tissue-specific expression control elements include promoters and enhancers active in hyperproliferative cells, such as cell proliferative disorders including neoplasias, tumors and cancers, and metastasis. Particular non-limiting examples of such promoters are hexokinase II, COX-2, alpha-fetoprotein, carcinoembryonic antigen, DE3/MUC1, prostate specific antigen, C-erB2/neu, telomerase reverse transcriptase and hypoxia-responsive promoter.

For bacterial expression, constitutive promoters include T7, as well as inducible promoters such as pL of bacteriophage λ, plac, ptrp, ptac (ptrp-lac hybrid promoter). In insect cell systems, constitutive or inducible promoters (e.g., ecdysone) may be used. In yeast, constitutive promoters include, for example, ADH or LEU2 and inducible promoters such as GAL (see, e.g., Ausubel et al., In: Current Protocols in Molecular Biology, Vol. 2, Ch. 13, ed., Greene Publish. Assoc. & Wiley Interscience, 1988; Grant et al., In: Methods in Enzymology, 153:516-544 (1987), eds. Wu & Grossman, 1987, Acad. Press, N.Y.; Glover, DNA Cloning, Vol. II, Ch. 3, IRL Press, Wash., D.C., 1986; Bitter, In: Methods in Enzymology, 152:673-684 (1987), eds. Berger & Kimmel, Acad. Press, N.Y.; and, Strathem et al., The Molecular Biology of the Yeast Saccharomyces eds. Cold Spring Harbor Press, Vols. I and II (1982)).

For mammalian expression, constitutive promoters of viral or other origins may be used. For example, SV40, or viral long terminal repeats (LTRs) and the like, or inducible promoters derived from the genome of mammalian cells (e.g., metallothionein IIA promoter; heat shock promoter, steroid/thyroid hormone/retinoic acid response elements) or from mammalian viruses (e.g., the adenovirus late promoter; mouse mammary tumor virus LTR) are used.

In accordance with the invention, there are provided host cells transformed or transfected with nucleic acids and vectors of the invention. In one embodiment, a cell is stably or transiently transformed with a nucleic acid that encodes an antibody, a functional fragment, a heavy or light chain sequence, or a portion of a heavy or light chain sequence (e.g., a variable region, or one or more CDRs). In another embodiment, a host cell is stably or transiently transformed with an antisense or inhibitory nucleic acid.

Host cells include but are not limited to prokaryotic and eukaryotic cells such as bacteria, fungi (yeast), plant, insect, and animal (e.g., mammalian, including primate and human) cells. The cells may be a primary cell isolate, cell culture (e.g., passaged, established or immortalized cell line), or part of a plurality of cells, or a tissue or organ ex vivo or in a subject (in vivo). For example, bacteria transformed with recombinant bacteriophage nucleic acid, plasmid nucleic acid or cosmid nucleic acid expression vectors; yeast transformed with recombinant yeast expression vectors; plant cell systems infected with recombinant virus expression vectors (e.g., cauliflower mosaic virus, CaMV; tobacco mosaic virus, TMV) or transformed with recombinant plasmid expression vectors (e.g., Ti plasmid); insect cell systems infected with recombinant virus expression vectors (e.g., baculovirus); and animal cell systems infected with recombinant virus expression vectors (e.g., retroviruses, adenovirus, vaccinia virus), or transformed animal cell systems engineered for stable expression.

The term “transformed” or “transfected” when use in reference to a cell (e.g., a host cell) or organism, means a genetic change in a cell following incorporation of an exogenous molecule, for example, a protein or nucleic acid (e.g., a transgene) into the cell. Thus, a “transfected” or “transformed” cell is a cell into which, or a progeny thereof in which an exogenous molecule has been introduced by the hand of man, for example, by recombinant DNA techniques.

The nucleic acid can be stably or transiently transfected or transformed (expressed) in the cell and progeny thereof. Host cells therefore include those that stably or transiently express antibody, functional fragment or nucleic acid. The cell(s) can be propagated and the introduced antibody expressed, or nucleic acid transcribed. A progeny of a transfected or transformed cell may not be identical to the parent cell, since there may be mutations that occur during replication.

Typically, cell transfection or transformation employs a “vector,” which refers to a plasmid, virus, such as a viral vector, or other vehicle known in the art that can be manipulated by insertion or incorporation of a nucleic acid.

A viral particle or vesicle can be designed to be targeted to particular cell types (e.g., hyperproliferating cells) by inclusion of a protein on the surface that binds to a target cell ligand or receptor. Alternatively, a cell type-specific promoter and/or enhancer can be included in the vector in order to express the nucleic acid in target cells. Thus, the viral particle or vesicle itself, viral vector, or a protein on the viral surface can be made to target cells for transfection or transformation in vitro, ex vivo or in vivo.

Introduction of compositions (e.g., protein and nucleic acid) into target cells (e.g., host cells) can also be carried out by methods known in the art such as osmotic shock (e.g., calcium phosphate), electroporation, microinjection, cell fusion, etc. Introduction of nucleic acid and polypeptide in vitro, ex vivo and in vivo can also be accomplished using other techniques. For example, a polymeric substance, such as polyesters, polyamine acids, hydrogel, polyvinyl pyrrolidone, ethylene-vinylacetate, methylcellulose, carboxymethylcellulose, protamine sulfate, or lactide/glycolide copolymers, polylactide/glycolide copolymers, or ethylenevinylacetate copolymers. A nucleic acid can be entrapped in microcapsules prepared by coacervation techniques or by interfacial polymerization, for example, by the use of hydroxymethylcellulose or gelatin-microcapsules, or poly (methylmethacrolate) microcapsules, respectively, or in a colloid system. Colloidal dispersion systems include macromolecule complexes, nano-capsules, microspheres, beads, and lipid-based systems, including oil-in-water emulsions, micelles, mixed micelles, and liposomes.

Liposomes for introducing various compositions into cells are known in the art and include, for example, phosphatidylcholine, phosphatidylserine, lipofectin and DOTAP (e.g., U.S. Pat. Nos. 4,844,904, 5,000,959, 4,863,740, and 4,975,282; and GIBCO-BRL, Gaithersburg, Md.). Piperazine based amphilic cationic lipids useful for gene therapy also are known (see, e.g., U.S. Pat. No. 5,861,397). Cationic lipid systems also are known (see, e.g., U.S. Pat. No. 5,459,127). Polymeric substances, microcapsules and colloidal dispersion systems such as liposomes are collectively referred to herein as “vesicles.” Accordingly, viral and non-viral vector means of delivery into cells, tissue or organs, in vitro, in vivo and ex vivo are included.

The invention includes in vivo methods. For example, a cell such as an undesirably proliferating cell or cell proliferative disorder to which BARB3 antibody or functional fragment binds can be present in a subject, such as a mammal (e.g., a human subject). A subject having such cells may therefore be treated by administering, for example, an antibody, or subsequence or fragment thereof, that binds to such cells.

In accordance with the invention, there are provided methods of treating undesirable cell proliferation or a cell proliferative or cellular hyperproliferative disorder in a subject. Such methods can be practiced with any of the antibodies, functional fragments, modified and variant forms set forth herein. In one embodiment, a method includes administering to a subject an amount of BARB3 antibody, as represented by DSMZ Deposit No. DSM ACC2859, or heavy and light chain sequences set forth as SEQ ID NOs:1 and 2, effective to treat the undesirable cell proliferation or a cell proliferative or cell hyperproliferative disorder in the subject.

As used herein, the terms “cell proliferative disorder” and “cellular hyperproliferative disorder” and grammatical variations thereof, when used in reference to a cell, tissue or organ, refers to any undesirable, excessive or abnormal cell, tissue or organ growth, proliferation, differentiation or survival. A hyperproliferative cell denotes a cell whose growth, proliferation, or survival is greater than desired, such as a reference normal cell, e.g., a cell that is of the same tissue or organ but is not a hyperproliferative cell, or a cell that fails to differentiate normally. Undesirable cell proliferation and hyperproliferative disorders include diseases and physiological conditions, both benign hyperplastic conditions characterized by undesirable, excessive or abnormal cell numbers, cell growth, cell proliferation, cell survival or differentiation in a subject. Specific examples of such disorders include metastatic and non-metastatic neoplasia, tumors and cancers (malignancies).

In various embodiments, a method includes administering to a subject a BARB3 antibody, as represented by DSMZ Deposit No. DSM ACC2859, or heavy and light chain sequences set forth as SEQ ID NOs:1 and 2, in an amount effective to treat the cell proliferative or cellular hyperproliferative disorder in the subject. In particular aspects, the disorder is a neoplasia, tumor or metastatic or non-metastatic cancer (malignancy). In additional aspects, the disorder affects or is present in part at least in breast, lung, thyroid, head and neck, nasopharynx, nose or sinuses, brain, spine, adrenal gland, thyroid, lymph, gastrointestinal (mouth, esophagus, stomach, duodenum, ileum, jejunum (small intestine), colon, rectum), genito-urinary tract (uterus, ovary, cervix, bladder, testicle, penis, prostate), kidney, pancreas, adrenal gland, liver, bone, bone marrow, lymph, blood, muscle, skin, or the hematopoetic system.

The terms “tumor,” “cancer” and “neoplasia” are used interchangeably and refer to a cell or population of cells whose growth, proliferation or survival is greater than growth, proliferation or survival of a normal counterpart cell, e.g. a cell proliferative or differentiative disorder. Typically, the growth is uncontrolled. The term “malignancy” refers to invasion of nearby tissue. The term “metastasis” refers to spread or dissemination of a tumor, cancer or neoplasia to other sites, locations or regions within the subject, in which the sites, locations or regions are distinct from the primary tumor or cancer.

Invention methods can be used to reduce or inhibit metastasis of a primary tumor or cancer to other sites, or the formation or establishment of metastatic tumors or cancers at other sites distal from the primary tumor or cancer thereby inhibiting or reducing tumor or cancer relapse or tumor or cancer progression. Thus, methods of the invention include, among other things, 1) reducing or inhibiting growth, proliferation, mobility or invasiveness of tumor or cancer cells that potentially or do develop metastases (e.g, disseminated tumor cells, DTC); 2) reducing or inhibiting formation or establishment of metastases arising from a primary tumor or cancer to one or more other sites, locations or regions distinct from the primary tumor or cancer; 3) reducing or inhibiting growth or proliferation of a metastasis at one or more other sites, locations or regions distinct from the primary tumor or cancer after a metastasis has formed or has been established; and 4) reducing or inhibiting formation or establishment of additional metastasis after the metastasis has been formed or established.

Neoplasias, tumors and cancers include a sarcoma, carcinoma, adenocarcinoma, melanoma, myeloma, blastoma, glioma, lymphoma or leukemia. Exemplary cancers include, for example, carcinoma, sarcoma, adenocarcinoma, melanoma, neural (blastoma, glioma), mesothelioma and reticuloendothelial, lymphatic or haematopoietic neoplastic disorders (e.g., myeloma, lymphoma or leukemia). In particular aspects, a neoplasia, tumor or cancer includes a lung adenocarcinoma, lung carcinoma, diffuse or interstitial gastric carcinoma, colon adenocarcinoma, prostate adenocarcinoma, esophagus carcinoma, breast carcinoma, pancreas adenocarcinoma, ovarian adenocarcinoma, or uterine adenocarcinoma.

Neoplasia, tumors and cancers include benign, malignant, metastatic and non-metastatic types, and include any stage (I, II, III, IV or V) or grade (G1, G2, G3, etc.) of neoplasia, tumor, or cancer, or a neoplasia, tumor, cancer or metastasis that is progressing, worsening, stabilized or in remission.

Neoplasias, tumors and cancers can arise from a multitude of primary tumor types, including but not limited to breast, lung, thyroid, head and neck, nasopharynx, nose or sinuses, brain, spine, adrenal gland, thyroid, lymph, gastrointestinal (mouth, esophagus, stomach, duodenum, ileum, jejunum (small intestine), colon, rectum), genito-urinary tract (uterus, ovary, cervix, bladder, testicle, penis, prostate), kidney, pancreas, adrenal gland, liver, bone, bone marrow, lymph, blood, muscle, skin, and the hematopoetic system, and may metastasize to secondary sites.

A “solid neoplasia, tumor or cancer” refers to neoplasia, tumor or cancer (e.g., metastasis) that typically aggregates together and forms a mass. Specific examples include visceral tumors such as melanomas, breast, pancreatic, uterine and ovarian cancers, testicular cancer, including seminomas, gastric or colon cancer, hepatomas, adrenal, renal and bladder carcinomas, lung, head and neck cancers and brain tumors/cancers.

Carcinomas refer to malignancies of epithelial or endocrine tissue, and include respiratory system carcinomas, gastrointestinal system carcinomas, genitourinary system carcinomas, testicular carcinomas, breast carcinomas, prostatic carcinomas, endocrine system carcinomas, and melanomas. The term also includes carcinosarcomas, e.g., which include malignant tumors composed of carcinomatous and sarcomatous tissues. Adenocarcinoma includes a carcinoma of a glandular tissue, or in which the tumor forms a gland like structure. Melanoma refers to malignant tumors of melanocytes and other cells derived from pigment cell origin that may arise in the skin, the eye (including retina), or other regions of the body. Additional carcinomas can form from the uterine/cervix, lung, head/neck, colon, pancreas, testes, adrenal gland, kidney, esophagus, stomach, liver and ovary.

Sarcomas refer to malignant tumors of mesenchymal cell origin. Exemplary sarcomas include for example, lymphosarcoma, liposarcoma, osteosarcoma, chondrosarcoma, leiomyosarcoma, rhabdomyosarcoma and fibrosarcoma.

Neural neoplasias include glioma, glioblastoma, meningioma, neuroblastoma, retinoblastoma, astrocytoma, oligodendrocytoma

Specific non-limiting examples of neoplasias, tumors and cancers amenable to treatment include malignant and non-malignant neoplasias, tumors and cancers, and metastasis. In particular, gastric (stomach) tissue, lung squamous cell carcinoma, and lung adenocarcinoma cell of any stage (e.g., stages IA, IB, IIA, IIB, IIIA, IIIB or IV) or grade (e.g., grades G1, G2 or G3). Additional non-limiting examples include adenocarcinoma or a squamous cell carcinoma, such as a stomach adenocarcinoma, a lung adenocarcinoma, a pancreas adenocarcinoma, a colon adenocarcinoma, a breast adenocarcinoma, or an esophagus squamous cell carcinoma. Further non-limiting examples include a human adenocarcinoma, human squamous cell carcinoma, human carcinoid carcinoma, human invasive ductal carcinoma, human germ cell carcinoma in any of stomach, lung, colon, pancreas, esophagus, prostate, breast or testis.

A “liquid neoplasia, tumor or cancer” refers to a neoplasia, tumor or cancer of the reticuloendothelial or hematopoetic system, such as a lymphoma, myeloma, or leukemia, or a neoplasia that is diffuse in nature. Particular examples of leukemias include acute and chronic lymphoblastic, myeloblastic and multiple myeloma. Typically, such diseases arise from poorly differentiated acute leukemias, e.g., erythroblastic leukemia and acute megakaryoblastic leukemia. Specific myeloid disorders include, but are not limited to, acute promyeloid leukemia (APML), acute myelogenous leukemia (AML) and chronic myelogenous leukemia (CML); lymphoid malignancies include, but are not limited to, acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Specific malignant lymphomas include, non-Hodgkin lymphoma and variants, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

As used herein, the terms “treat,” “treating,” “treatment” and grammatical variations thereof mean subjecting an individual patient to a protocol, regimen, process or remedy, in which it is desired to obtain a physiologic response or outcome in that patient. Since every treated patient may not respond to a particular treatment protocol, regimen, process or remedy, treating does not require that the desired physiologic response or outcome be achieved in each and every patient or patient population. Accordingly, a given patient or patient population may fail to respond or respond inadequately to treatment.

Methods of the invention may be practiced by any mode of administration or by any route, systemic, regional and local administration. Exemplary administration routes include intravenous, intrarterial, intradermal, intramuscular, subcutaneous, intra-pleural, transdermal (topical), transmucosal, intra-cranial, intra-spinal, intra-ocular, rectal, oral (alimentary) and mucosal.

Methods of the invention include, among other things, methods that provide a detectable or measurable improvement in a condition of a given subject, such as alleviating or ameliorating one or more adverse (physical) symptoms or consequences associated with the presence of a cell proliferative or cellular hyperproliferative disorder, neoplasia, tumor or cancer, or metastasis, i.e., a therapeutic benefit or a beneficial effect.

A therapeutic benefit or beneficial effect is any objective or subjective, transient, temporary, or long-term improvement in the condition or pathology, or a reduction in onset, severity, duration or frequency of an adverse symptom associated with or caused by cell proliferation or a cellular hyperproliferative disorder such as a neoplasia, tumor or cancer, or metastasis. A satisfactory clinical endpoint of a treatment method in accordance with the invention is achieved, for example, when there is an incremental or a partial reduction in severity, duration or frequency of one or more associated pathologies, adverse symptoms or complications, or inhibition or reversal of one or more of the physiological, biochemical or cellular manifestations or characteristics of cell proliferation or a cellular hyperproliferative disorder such as a neoplasia, tumor or cancer, or metastasis. A therapeutic benefit or improvement therefore be a cure, such as destruction of target proliferating cells (e.g., neoplasia, tumor or cancer, or metastasis) or ablation of one or more, most or all pathologies, adverse symptoms or complications associated with or caused by cell proliferation or the cellular hyperproliferative disorder such as a neoplasia, tumor or cancer, or metastasis. However, a therapeutic benefit or improvement need not be a cure or complete destruction of all target proliferating cells (e.g., neoplasia, tumor or cancer, or metastasis) or ablation of all pathologies, adverse symptoms or complications associated with or caused by cell proliferation or the cellular hyperproliferative disorder such as a neoplasia, tumor or cancer, or metastasis. For example, partial destruction of a tumor or cancer cell mass, or a stabilization of the tumor or cancer mass, size or cell numbers by inhibiting progression or worsening of the tumor or cancer, can reduce mortality and prolong lifespan even if only for a few days, weeks or months, even though a portion or the bulk of the tumor or cancer mass, size or cells remain.

Specific non-limiting examples of therapeutic benefit include a reduction in neoplasia, tumor or cancer, or metastasis volume (size or cell mass) or numbers of cells, inhibiting or preventing an increase in neoplasia, tumor or cancer volume (e.g., stabilizing), slowing or inhibiting neoplasia, tumor or cancer progression, worsening or metastasis, stimulating, inducing or increasing neoplasia, tumor or cancer cell lysis or apoptosis or inhibiting neoplasia, tumor or cancer proliferation, growth or metastasis. An invention method may not take effect immediately. For example, treatment may be followed by an increase in the neoplasia, tumor or cancer cell numbers or mass, but over time eventual stabilization or reduction in tumor cell mass, size or numbers of cells in a given subject may subsequently occur after cell lysis or apoptosis of the neoplasia, tumor or cancer, or metastasis.

Additional adverse symptoms and complications associated with neoplasia, tumor, cancer and metastasis that can be inhibited, reduced, decreased, delayed or prevented include, for example, nausea, lack of appetite, lethargy, pain and discomfort. Thus, a partial or complete decrease or reduction in the severity, duration or frequency of an adverse symptom or complication associated with or caused by a cellular hyperproliferative disorder, an improvement in the subjects well being, such as increased energy, appetite, psychological well being, are all particular non-limiting examples of therapeutic benefit. A therapeutic benefit or improvement therefore can also include a subjective improvement in the quality of life of a treated subject.

In various embodiments, a method reduces or decreases neoplasia, tumor or cancer, or metastasis volume, inhibits or prevents an increase in neoplasia, tumor or cancer volume, inhibits or delays neoplasia, tumor or cancer progression or worsening, stimulates neoplasia, tumor or cancer, or metastasis cell lysis or apoptosis, or inhibits, reduces, decreases or delays neoplasia, tumor or cancer proliferation or metastasis. In an additional embodiment, a method prolongs or extends lifespan of the subject. In a further embodiment, a method improves the quality of life of the subject.

Examination of a biopsied sample containing a neoplasia, tumor or cancer, or metastasis (e.g., blood or tissue sample), can establish neoplastic, tumor or cancer cell volume or cell numbers, and therefore whether a reduction or stabilization in mass or numbers of neoplastic, tumor or cancer cells or inhibition of neoplasia, tumor or cancer cell proliferation, growth or survival (apoptosis) has occurred. For a solid neoplasia, tumor or cancer, invasive and non-invasive imaging methods can ascertain neoplasia, tumor or cancer size or volume. Examination of blood or serum, for example, for populations, numbers and types of cells (e.g., hematopoetic cellular hyperproliferative disorders) can establish whether a reduction or stabilization in mass or numbers of neoplastic, tumor or cancer cells or inhibition of neoplastic, tumor or cancer proliferation, growth or survival (apoptosis) has occurred.

Invention compositions and methods can be combined with any other treatment or therapy that provides a desired effect. In particular, treatments and therapies that have been characterized as having an anti-cell proliferative activity or function are applicable. Exemplary treatments and therapies include anti-cell proliferative or immune enhancing agents or drugs.

The treatments and therapies can be performed prior to, substantially contemporaneously with any other methods of the invention, for example, an anti-cell proliferative or anti-cellular hyperproliferative disorder (e.g., a neoplasia, tumor or cancer, or metastasis).

The invention therefore provides combination methods in which the methods of the invention, in which any of the antibodies, functional fragments, and modified and variant forms, are used in a combination with any therapeutic regimen, treatment protocol or composition, such as an anti-cell proliferative protocol, agent or drug set forth herein or known in the art. In one embodiment, a method includes administering BARB3 antibody, as represented by DSMZ Deposit No. DSM ACC2859, or heavy and light chain sequences set forth as SEQ ID NOs:1 and 2, and an anti-cell proliferative or immune enhancing treatment, agent or drug. The anti-cell proliferative or immune enhancing treatment, agent or drug can be administered prior to, substantially contemporaneously with or following administration of BARB3 antibody, as represented by DSMZ Deposit No. DSM ACC2859, or heavy and light chain sequences set forth as SEQ ID NOs:1 and 2.

As used herein, an “anti-cell proliferative,” “anti-neoplastic,” “anti-tumor,” or “anti-cancer” treatment, therapy, activity or effect means any therapy, treatment regimen, agent, drug, protocol or process that is useful in treating pathologies, adverse symptoms or complications associated with or caused by abnormal or undesirable cell proliferation (cell hyperproliferation), a cellular hyperproliferative disorder, neoplasia, tumor or cancer, or metastasis. Particular therapies, treatment regimens, agents, drugs, protocol or processes can inhibit, decrease, slow, reduce, delay, or prevent cell proliferation, cell growth, cellular hyperproliferation, neoplastic, tumor, or cancer (malignant) growth, proliferation, survival or metastasis. Such treatments, therapies, regimens, protocols, agents and drugs, can operate by disrupting, reducing, inhibiting or delaying cell cycle progression or cell proliferation or growth; increasing, stimulating or enhancing cell apoptosis, lysis or death; inhibiting nucleic acid or protein synthesis or metabolism; reducing, decreasing, inhibiting or delaying cell division; or decreasing, reducing or inhibiting cell survival, or production or utilization of a cell survival factor, growth factor or signaling pathway (extracellular or intracellular).

Examples of anti-cell proliferative treatments and therapies include chemotherapy, immunotherapy, radiotherapy (ionizing or chemical), local or regional thermal (hyperthermia) therapy and surgical resection.

Specific non-limiting classes of anti-cell proliferative agents and drugs include alkylating agents, anti-metabolites, plant extracts, plant alkaloids, nitrosoureas, hormones (steroids), nucleoside and nucleotide analogues. Specific non-limiting examples of microbial toxins include bacterial cholera toxin, pertussis toxin, anthrax toxin, diphtheria toxin, and plant toxin ricin. Specific examples of drugs include cyclophosphamide, azathioprine, cyclosporin A, melphalan, chlorambucil, mechlorethamine, busulphan, methotrexate, 6-mercaptopurine, thioguanine, 5-fluorouracil, 5-fluorouridine, cytosine arabinoside, AZT, 5-azacytidine (5-AZC) and 5-azacytidine related compounds, bleomycin, actinomycin D, mithramycin, mitomycin C, carmustine, calicheamicin, lomustine, semustine, streptozotocin, teniposide, etoposide, hydroxyurea, cisplatin, carboplatin, levamisole, mitotane, procarbazine, dacarbazine, taxol, vinblastine, vincristine, vindesine, doxorubicin, daunomycin and dibromomannitol. Specific non-limiting examples of hormones include prednisone, prednisolone, diethylstilbesterol, flutamide, leuprolide, and gonatrophin releasing hormone antagonists.

Radiotherapy includes internal or external delivery to a subject. For example, alpha, beta, gamma and X-rays can administered to the subject externally without the subject internalizing or otherwise physically contacting the radioisotope. Specific examples of X-ray dosages range from daily doses of 50 to 200 roentgens for prolonged periods of time (3 to 5/week), to single doses of 2000 to 6000 roentgens. Dosages vary widely, and depend on duration of exposure, the half-life of the isotope, the type of radiation emitted, the cell type and location treated and the progressive stage of the disease. Specific non-limiting examples of radionuclides include, for example, ⁴⁷Sc ⁶⁷Cu, ⁷²Se, ⁸⁸Y, ⁹⁰Sr, ⁹⁰Y, ⁹⁷Ru, ⁹⁹Tc, ¹⁰⁵Rh, ¹¹¹In, ¹²⁵I, ¹³¹I, ¹⁴⁹Tb, ¹⁵³Sm, ¹⁸⁶Re, ¹⁸⁸Re, ¹⁹⁴Os, ²⁰³Pb, ²¹¹At, ²¹²Bi, ²¹³Bi, ²¹²Pb, ²²³Ra, ²²⁵Ac, ²²⁷Ac, and ²²⁸Th.

Antibodies that bind to tumor cells are a particular example of an anti-cell proliferative treatment or therapy. Anti-tumor antibodies include, for example, M195 antibody which binds to leukemia cell CD33 antigen (U.S. Pat. No. 6,599,505); monoclonal antibody DS6 which binds to ovarian carcinoma CA6 tumor-associated antigen (U.S. Pat. No. 6,596,503); human IBD12 monoclonal antibody which binds to epithelial cell surface H antigen (U.S. Pat. No. 4,814,275); and BR96 antibody which binds to Le^{x} carbohydrate epitope expressed by colon, breast, ovary, and lung carcinomas. Additional anti-tumor antibodies that can be employed include, for example, Herceptin (anti-Her-2 neu antibody), Rituxan®, Zevalin, Bevacizumab (Avastin), Bexxar, Campath®, Oncolym, 17-1A (Edrecolomab), 3F8 (anti-neuroblastoma antibody), MDX-CTLA4, IMC-C225 (Cetuximab) and Mylotarg.

As used here, the term “immune enhancing,” when used in reference to a treatment, therapy, agent or drug means that the treatment, therapy, agent or drug provides an increase, stimulation, induction or promotion of an immune response, humoral or cell-mediated. Such therapies can enhance immune response generally, or enhance immune response to a specific target, e.g., a cell proliferative or cellular hyperproliferative disorder such as a neoplasia, tumor or cancer, or metastasis.

Specific non-limiting examples of immune enhancing agents include antibody, cell growth factors, cell survival factors, cell differentiative factors, cytokines and chemokines. Additional examples of immune enhancing agents and treatments include immune cells such as lymphocytes, plasma cells, macrophages, dendritic cells, NK cells and B-cells that either express antibody against the cell proliferative disorder or otherwise are likely to mount an immune response against the cell proliferative disorder. Cytokines that enhance or stimulate immunogenicity include IL-2, IL-1α, IL-1, IL-3, IL-6, IL-7, granulocyte-macrophage-colony stimulating factor (GMCSF), IFN-γ, IL-12, TNF-α, and TNFβ, which are also non-limiting examples of immune enhancing agents. Chemokines including MIP-1α, MIP-1β, RANTES, SDF-1, MCP-1, MCP-2, MCP-3, MCP-4, eotaxin, eotaxin-2, I-309/TCA3, ATAC, HCC-1, HCC-2, HCC-3, PARC, TARC, LARC/MIP-3α, CKβ, CKβ6, CKβ7, CKβ8, CKβ9, CKβ11, CKβ12, C10, IL-8, ENA-78, GROα, GROβ, GCP-2, PBP/CTAPIIIβ-TG/NAP-2, Mig, PBSF/SDF-1, and lymphotactin are further non-limiting examples of immune enhancing agents.

Methods of the invention also include, among other things, methods that result in a reduced need or use of another treatment protocol or therapeutic regimen, process or remedy. For example, for a neoplasia, tumor or cancer, or metastasis, a method of the invention has a therapeutic benefit if in a given subject it results in a less frequent or reduced dose or elimination of an anti-cell proliferative (e.g., anti-neoplastic, anti-tumor or anti-cancer) or immune enhancing treatment or therapy, such as a chemotherapeutic drug, radiotherapy, immunotherapy, or surgery for neoplasia, tumor or cancer, or metastasis treatment or therapy.

In accordance with the invention, methods of reducing need or use of an anti-cell proliferative (e.g., anti-neoplastic, anti-tumor, anti-cancer or anti-metastasis) treatment or therapy are provided. In one embodiment, a method includes administering to a subject BARB3 antibody, as represented by DSMZ Deposit No. DSM ACC2859, or heavy and light chain sequences set forth as SEQ ID NOs:1 and 2, in an amount effective to treat a cellular hyperproliferative disorder (e.g., a neoplasia, tumor or cancer, or metastasis), and to reduce or eliminate need for an anti-cell proliferative (anti-neoplasia, anti-tumor or anti-cancer, or anti-metastasis) or immune-enhancing therapy. The methods can be performed prior to, substantially contemporaneously with or following administration of an anti-neoplastic, tumor, cancer or metastasis, or immune-enhancing therapy.

The doses or “amount effective” or “amount sufficient” in a method of treatment or therapy in which it is desired to achieve a therapeutic benefit or improvement includes, for example, any objective or subjective alleviation or amelioration of one, several or all pathologies, adverse symptoms or complications associated with or caused by the target (e.g., cellular hyperproliferative disorder), to a measurable or detectable extent, although preventing, inhibiting or delaying a progression or worsening of the target (e.g., cellular hyperproliferative disorder) pathology, adverse symptom or complication, is a satisfactory outcome. Thus, in the case of a cellular hyperproliferative disorder, the amount will be sufficient to provide a therapeutic benefit to a given subject or to alleviate or ameliorate a pathology, adverse symptom or complication of the disorder in a given subject. The dose may be proportionally increased or reduced as indicated by the status of treatment or therapeutic target (e.g., cellular hyperproliferative disorder) or any side effect(s) of the treatment or therapy.

Exemplary non-limiting amounts (doses) are in a range of about 0.1 mg/kg to about 100 mg/kg, and any numerical value or range or value within such ranges. Greater or lesser amounts (doses) can be administered, for example, 0.01-500 mg/kg, and any numerical value or range or value within such ranges. Additional exemplary non-limiting amounts (doses) range from about 0.5-50 mg/kg, 1.0-25 mg/kg, 1.0-10 mg/kg, and any numerical value or range or value within such ranges.

Methods of the invention may be practiced one or more times (e.g., 1-10, 1-5 or 1-3 times) per day, week, month, or year. The skilled artisan will know when it is appropriate to delay or discontinue administration. An exemplary non-limiting dosage schedule is 1-7 times per week, for 1, 23, 4, 5, 6, 7, 8, 9, 10, 15, 20 or more weeks, and any numerical value or range or value within such ranges.

Of course, as is typical for any treatment or therapy, different subjects will exhibit different responses to treatment and some may not respond or respond inadequately to a particular treatment protocol, regimen or process. Amounts effective or sufficient will therefore depend at least in part upon the disorder treated (e.g., cell proliferation, benign hyperplasia or a neoplasia, tumor or cancer and the type or stage, e.g., the tumor or cancer grade and if it is advanced, late or early stage), the therapeutic effect desired, as well as the individual subject (e.g., the bioavailability within the subject, gender, age, etc.) and the subject's response to the treatment based upon genetic and epigenetic variability (e.g., pharmacogenomics).

Cell toxicity and viability (cell apoptosis, lysis, growth proliferation, etc.) can be measured in a variety of ways on the basis of calorimetric, luminescent, radiometric, or fluorometric assays known in the art. Colorimetric techniques for determining cell viability include, for example, Trypan Blue exclusion (see, for example, Examples 1 and 2). In brief, cells are stained with Trypan Blue and counted using a hemocytometer. Viable cells exclude the dye whereas dead and dying cells take up the blue dye and are easily distinguished under a light microscope. Neutral Red is adsorbed by viable cells and concentrates in cell lysosomes; viable cells can be determined with a light microscope by quantitating numbers of Neutral Red stained cells.

Fluorometric techniques for determining cell viability include, for example, propidium iodide, a fluorescent DNA intercalating agent. Propidium iodide is excluded from viable cells but stains the nucleus of dead cells. Flow cytometry of propidium iodide labeled cells can then be used to quantitate viable and dead cells. Release of lactate dehydrogenase (LDH) indicates structural damage and death of cells, and can be measured by a spectrophotometric enzyme assay. Bromodeoxyuridine (BrdU) is incorporated into newly synthesized DNA and can be detected with a fluorochrome-labeled antibody. The fluorescent dye Hoechst 33258 labels DNA and can be used to quantitate proliferation of cells (e.g., flow cytometry). Quantitative incorporation of the fluorescent dye carboxyfluorescein diacetate succinimidyl ester (CFSE or CFDA-SE) can provide cell division analysis (e.g., flow cytometry). This technique can be used either in vitro or in vivo. 7-aminoactinomycin D (7-AAD) is a fluorescent intercalator that undergoes a spectral shift upon association with DNA, and can provide cell division analysis (e.g., flow cytometry).

Radiometric techniques for determining cell proliferation include, for example, [³H]-Thymidine, which is incorporated into newly synthesized DNA of living cells and frequently used to determine proliferation of cells. Chromium (⁵¹Cr)-release from dead cells can be quantitated by scintillation counting in order to quantitate cell viability.

Luminescent techniques for determining cell viability include, for example, the CellTiter-Glo luminescent cell viability assay (Promega Madison Wis.). This technique quantifies the amount of ATP present to determine the number of viable cells.

Commercially available kits for determining cell viability and cell proliferation include, for example, Cell Proliferation Biotrak ELISA (Amersham Biosciences Piscataway, N.J.); the Guava ViaCount™ Assay, which provides rapid cell counts and viability determination based on differential uptake of fluorescent reagents (Guava Technologies, Hayward, Calif.); the CyQUANT® Cell Proliferation Assay Kit (Molecular Probes, Inc., Eugene, Oreg.); and the CytoLux Assay Kit (PerkinElmer Life Sciences Inc., Boston, Mass.). The DELFIA® Assay Kits (PerkinElmer Life Sciences Inc., Boston, Mass.) can determine cell proliferation and viability using a time-resolved fluorometric method. The Quantos™ Cell Proliferation Assay is a fluorescence-based assay that measures the fluorescence of a DNA-dye complex from lysed cells (Stratagene, La Jolla, Calif.). The CellTiter-Glo cell viability assay is a luminescent assay for measuring cell viability (Promega, Madison Wis.).

The terms “subject” and “patient” are used interchangeably herein and refer to animals, typically mammals, such as humans, non-human primates (gorilla, chimpanzee, orangutan, macaque, gibbon), domestic animals (dog and cat), farm and ranch animals (horse, cow, goat, sheep, pig), laboratory and experimental animals (mouse, rat, rabbit, guinea pig). Subjects include disease model animals (e.g. such as mice, rats and non-human primates) for studying in vivo efficacy (e.g., a neoplasia, tumor or cancer, or metastasis animal model). Human subjects include children, for example, newborns, infants, toddlers and teens, between the ages of 1 and 5, 5 and 10 and 10 and 18 years, adults between the ages of 18 and 60 years, and the elderly, for example, between the ages of 60 and 65, 65 and 70 and 70 and 100 years.

Subjects include mammals (e.g., humans) in need of treatment, that is, they have undesirable or aberrant cell proliferation (cell hyperproliferation) or a cellular hyperproliferative disorder. Subjects also include those at risk of having a undesirable cell proliferation or a cellular hyperproliferative disorder. Subjects further include a subject in need of an anti-cell proliferative or immune enhancing treatment or therapy due to a lab or clinical diagnosis warranting such treatment, subjects undergoing an anti-cell proliferative or immune enhancing therapy, and subjects having undergone an anti-cell proliferative or immune enhancing therapy and are at risk of relapse or recurrence.

At risk subjects include those with a family history, genetic predisposition, or who have suffered a previous affliction with a cell proliferative or cellular hyperproliferative disorder (e.g., a benign hyperplasia, neoplasia, tumor or cancer, or metastasis), and are at risk of relapse or recurrence. At risk subjects further include environmental exposure to carcinogens or mutagens, such as smokers, or those in an occupational (industrial, chemical, agricultural) setting. Such subjects at risk for developing a cell proliferative or cellular hyperproliferative disorder such as neoplasia, tumor or cancer can be identified with genetic screens for tumor associated genes, gene deletions or gene mutations. Subjects that lack Brca1 are at risk for developing breast cancer, for example. Subjects at risk for developing colon cancer have deleted or mutated tumor suppressor genes, such as adenomatous polyposis coli (APC), for example. At risk subjects having particular genetic predisposition towards cell proliferative disorders are known (see, e.g., The Genetic Basis of Human Cancer 2^{nd} ed. by Bert Vogelstein (Editor), Kenneth W. Kinzler (Editor) (2002) McGraw-Hill Professional; The Molecular Basis of Human Cancer. Edited by W B Coleman and G J Tsongalis (2001) Humana Press; and The Molecular Basis of Cancer. Mendelsohn et al., W B Saunders (1995)).

At risk subjects can therefore be treated in order to inhibit or reduce the likelihood of developing a cell proliferative or cellular hyperproliferative disorder, or after having been cured of a cell proliferative disorder, suffering a relapse or recurrence of the same or a different cell proliferative or cellular hyperproliferative disorder. The result of such treatment can be to reduce the risk of developing a cell proliferative or cellular hyperproliferative disorder, or to prevent a cell proliferative or cellular hyperproliferative disorder, or a pathology, adverse symptom or complication thereof in the treated at risk subject.

The invention further provides kits, including antibodies, functional fragments, modified and variants forms, nucleic acids, agents, drugs and pharmaceutical formulations, packaged into suitable packaging material, optionally in combination with instructions for using the kit components, e.g., instructions for performing a method of the invention. In one embodiment, a kit includes a BARB3 antibody, as represented by DSMZ Deposit No. DSM ACC2859, or heavy and light chain sequences set forth as SEQ ID NOs:1 and 2. In one aspect, the instructions are for treating undesirable cell proliferation or hyperproliferation, or a cellular hyperproliferative disorder. In another aspect, the instructions are for treating a neoplasia, tumor or cancer, or metastasis. In a further embodiment, a kit includes a BARB3 antibody, as represented by DSMZ Deposit No. DSM ACC2859, or heavy and light chain sequences set forth as SEQ ID NOs:1 and 2, and instructions for treating undesirable cell proliferation or hyperproliferation, or a cellular hyperproliferative disorder, and an anti-cell proliferative or immune enhancing treatment, agent or drug. In various aspects, a kit includes an anti-neoplastic, anti-cancer or anti-tumor agent. In still a further aspects, a kit includes an article of manufacture, for example, an article of manufacture for delivering the antibody or nucleic acid, anti-cell proliferative or immune enhancing treatment, agent or drug into a subject locally, regionally or systemically.

The term “packaging material” refers to a physical structure housing the components of the kit. The packaging material can maintain the components sterilely, and can be made of material commonly used for such purposes (e.g., paper, corrugated fiber, glass, plastic, foil, ampules, etc.). The label or packaging insert can include appropriate written instructions, for example, practicing a method of the invention, e.g., treating a cell proliferative or cellular hyperproliferative disorder, an assay for screening for, detecting or identifying a BARB3 antigen or a cell to which BARB3 antibody, as represented by DSMZ Deposit No. DSM ACC2859, or heavy and light chain sequences set forth as SEQ ID NOs:1 and 2, binds, etc. Thus, in additional embodiments, a kit includes a label or packaging insert including instructions for practicing a method of the invention in solution, in vitro, in vivo, or ex vivo.

Instructions can therefore include instructions for practicing any of the methods of the invention described herein. For example, invention pharmaceutical compositions can be included in a container, pack, or dispenser together with instructions for administration to a subject to treat a cell proliferative or cellular hyperproliferative disorder, such as a neoplasia, tumor or cancer, or metastasis. Instructions may additionally include indications of a satisfactory clinical endpoint or any adverse symptoms or complications that may occur, storage information, expiration date, or any information required by regulatory agencies such as the Food and Drug Administration for use in a human subject.

The instructions may be on “printed matter,” e.g., on paper or cardboard within the kit, on a label affixed to the kit or packaging material, or attached to a vial or tube containing a component of the kit. Instructions may comprise voice or video tape and additionally be included on a computer readable medium, such as a disk (floppy diskette or hard disk), optical CD such as CD- or DVD-ROM/RAM, magnetic tape, electrical storage media such as RAM and ROM and hybrids of these such as magnetic/optical storage media.

Invention kits can additionally include a buffering agent, a preservative, or a protein/nucleic acid stabilizing agent. The kit can also include control components for assaying for activity, e.g., a control sample or a standard. Each component of the kit can be enclosed within an individual container or in a mixture and all of the various containers can be within single or multiple packages.

Antibodies (e.g., BARB3 antibody, as represented by DSMZ Deposit No. DSM ACC2859, or heavy and light chain sequences set forth as SEQ ID NOs:1 and 2) nucleic acids, and other compositions and methods of the invention can be included in or employ pharmaceutical formulations. Such pharmaceutical formulations are useful for treatment of, or administration or delivery to, a subject in vivo or ex vivo.

Pharmaceutical formulations include “pharmaceutically acceptable” and “physiologically acceptable” carriers, diluents or excipients. As used herein the terms “pharmaceutically acceptable” and “physiologically acceptable” include solvents (aqueous or non-aqueous), solutions, emulsions, dispersion media, coatings, isotonic and absorption promoting or delaying agents, compatible with pharmaceutical administration. Such formulations can be contained in a liquid; emulsion, suspension, syrup or elixir, or solid form; tablet (coated or uncoated), capsule (hard or soft), powder, granule, crystal, or microbead. Supplementary compounds (e.g., preservatives, antibacterial, antiviral and antifungal agents) can also be incorporated into the formulations.

Pharmaceutical formulations can be made to be compatible with a particular local, regional or systemic administration or delivery route. Thus, pharmaceutical formulations include carriers, diluents, or excipients suitable for administration by particular routes. Specific non-limiting examples of routes of administration for compositions of the invention are parenteral, e.g., intravenous, intrarterial, intradermal, intramuscular, subcutaneous, intra-pleural, transdermal (topical), transmucosal, intra-cranial, intra-spinal, intra-ocular, rectal, oral (alimentary), mucosal administration, and any other formulation suitable for the treatment method or administration protocol.

Solutions or suspensions used for parenteral application can include: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide.

Pharmaceutical formulations for injection include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (for example, glycerol, propylene glycol, and liquid polyetheylene glycol, and the like), and suitable mixtures thereof. Fluidity can be maintained, for example, by the use of a coating such as lecithin, by the maintenance of the required particle size in the case of dispersion and by the use of surfactants. Antibacterial and antifungal agents include, for example, parabens, chlorobutanol, phenol, ascorbic acid and thimerosal. Isotonic agents, for example, sugars, polyalcohols such as mannitol, sorbitol, sodium chloride can be included in the composition. Including an agent which delays absorption, for example, aluminum monostearate or gelatin can prolong absorption of injectable compositions.

Sterile injectable formulations can be prepared by incorporating the active composition in the required amount in an appropriate solvent with one or a combination of above ingredients. Generally, dispersions are prepared by incorporating the active composition into a sterile vehicle containing a basic dispersion medium and any other ingredient. In the case of sterile powders for the preparation of sterile injectable solutions, methods of preparation include, for example, vacuum drying and freeze-drying which yields a powder of the active ingredient plus any additional desired ingredient from a previously prepared solution thereof.

For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays, inhalation devices (e.g., aspirators) or suppositories. For transdermal administration, the active compounds are formulated into ointments, salves, gels, creams or patches.

The pharmaceutical formulations can be prepared with carriers that protect against rapid elimination from the body, such as a controlled release formulation or a time delay material such as glyceryl monostearate or glyceryl stearate. The formulations can also be delivered using articles of manufacture such as implants and microencapsulated delivery systems to achieve local, regional or systemic delivery or controlled or sustained release.

Biodegradable, biocompatible polymers can be used, such as ethylene vinyl acetate, polyanhydrides, polyglycolic acid, collagen, polyorthoesters, and polylactic acid. Methods for preparation of such formulations are known to those skilled in the art. The materials can also be obtained commercially from Alza Corporation (Palo Alto, Calif.). Liposomal suspensions (including liposomes targeted to cells or tissues using antibodies or viral coat proteins) can also be used as pharmaceutically acceptable carriers. These can be prepared according to known methods, for example, as described in U.S. Pat. No. 4,522,811.

Additional pharmaceutical formulations appropriate for administration are known in the art (see, e.g., Gennaro (ed.), Remington: The Science and Practice of Pharmacy, 20th ed., Lippincott, Williams & Wilkins (2000); Ansel et al., Pharmaceutical Dosage Forms and Drug Delivery Systems, 7^{th} ed., Lippincott Williams & Wilkins Publishers (1999); Kibbe (ed.), Handbook of Pharmaceutical Excipients American Pharmaceutical Association, 3^{rd} ed. (2000); and Remington's Pharmaceutical Principles of Solid Dosage Forms, Technonic Publishing Co., Inc., Lancaster, Pa., (1993)).

The compositions used in accordance with the invention, including proteins (antibodies), nucleic acid (inhibitory), treatments, therapies, agents, drugs and pharmaceutical formulations can be packaged in dosage unit form for ease of administration and uniformity of dosage. “Dosage unit form” as used herein refers to physically discrete units suited as unitary dosages treatment; each unit contains a quantity of the composition in association with the carrier, excipient, diluent, or vehicle calculated to produce the desired treatment or therapeutic (e.g., beneficial) effect. The unit dosage forms will depend on a variety of factors including, but not necessarily limited to, the particular composition employed, the effect to be achieved, and the pharmacodynamics and pharmacogenomics of the subject to be treated.

The invention provides cell-free (e.g., in solution, in solid phase) and cell-based (e.g., in vitro or in vivo) methods of screening, detecting and identifying a cell or antigen to which BARB3 antibody, as represented by DSMZ Deposit No. DSM ACC2859, or heavy and light chain sequences set forth as SEQ ID NOs:1 and 2, binds. The methods can be performed in solution, in vitro using a biological material or sample, and in vivo, for example, using neoplastic, tumor or cancer, or metastasis cells, tissue or organ (e.g., a biopsy) from an animal.

In accordance with the invention, there are provided methods of identifying, detecting or screening for a cell or antigen to which BARB3 antibody, as represented by DSMZ Deposit No. DSM ACC2859, or heavy and light chain sequences set forth as SEQ ID NOs:1 and 2, binds. In one embodiment, a method includes contacting a biological material or sample with a BARB3 antibody, as represented by DSMZ Deposit No. DSM ACC2859, or heavy and light chain sequences set forth as SEQ ID NOs:1 and 2, under conditions allowing binding of the antibody to a cell or antigen; and assaying for binding of the antibody to the cell or antigen. The binding of the antibody to a cell or antigen detects their presence. In one aspect, the biological material or sample is obtained from a mammalian subject. In a further aspect, the antibody that binds to the cell or antigen is distinct from BARB3 antibody, as represented by DSMZ Deposit No. DSM ACC2859, or heavy and light chain sequences set forth as SEQ ID NOs:1 and 2, binds. In an additional aspect, the cell expresses a cell membrane bound prohibitin isoform, or the antigen is a cell membrane bound prohibitin isoform antigen.

The invention also provides cell-free (e.g., in solution, in solid phase) and cell-based (e.g., in vitro or in vivo) methods of diagnosing a subject having or at increased risk of having undesirable or aberrant cell proliferation or a cellular hyperproliferative disorder (e.g., neoplasia, tumor or cancer, or metastasis). The methods can be performed in solution, in vitro using a biological material or sample, for example, a biopsy of suspicious cells that may comprise or be indicative of neoplastic, tumor or cancer, or metastasis cells, tissue or organ. The methods can also be preformed in vivo, for example, in an animal.

In accordance with the invention, there are provided methods of diagnosing a subject having or at increased risk of having undesirable or aberrant cell proliferation or a cellular hyperproliferative disorder (e.g., neoplasia, tumor or cancer, or metastasis). In one embodiment, a method includes providing a biological material or sample from a subject, contacting the biological material or sample with a BARB3 antibody, as represented by DSMZ Deposit No. DSM ACC2859, or heavy and light chain sequences set forth as SEQ ID NOs:1 and 2, under conditions allowing binding of the antibody to a cell or antigen; and assaying for binding of the antibody to the cell or antigen. The binding of the antibody to the cell or antigen diagnoses the subject as having or at increased risk of having undesirable or aberrant cell proliferation or a cellular hyperproliferative disorder (e.g., neoplasia, tumor or cancer, or metastasis). In one aspect, the biological material or sample is obtained from a human. In another aspect, the biological material or sample comprises a biopsy (e.g., a biopsy of lung, pancreas, stomach, breast, esophagus, ovary or uterus). In further aspects, the cell expresses prohibitin (e.g., a cell membrane bound prohibitin isoform, or SEQ ID NO:5), or the antigen comprises a prohibitin sequence (e.g., a cell membrane bound prohibitin isoform, or SEQ ID NO:5).

Identifying, detecting, screening and diagnostic assays of the invention can be practiced by analysis of suspect hyperproliferating cells, for example, a cell of a cellular hyperproliferative disorder. Cells include hyperproliferating, immortalized, neoplastic, tumor and cancer cell lines and primary isolates derived from breast, lung, thyroid, head and neck, nasopharynx, nose or sinuses, brain, spine, adrenal gland, thyroid, lymph, gastrointestinal (mouth, esophagus, stomach, duodenum, ileum, jejunum (small intestine), colon, rectum), genito-urinary tract (uterus, ovary, cervix, bladder, testicle, penis, prostate), kidney, pancreas, adrenal gland, liver, bone, bone marrow, lymph, blood, muscle, skin, and the hematopoetic system, and metastasis or secondary sites.

The term “contacting,” when used in reference to a composition such as a protein (e.g., antibody), material, sample, or treatment, means a direct or indirect interaction between the composition (e.g., protein such as an antibody) and the other referenced entity. A particular example of direct interaction is binding. A particular example of an indirect interaction is where the composition acts upon an intermediary molecule, which in turn acts upon the referenced entity. Thus, for example, contacting a cell (e.g., that comprises a cellular hyperproliferative disorder) with an antibody includes allowing the antibody to bind to the cell, or allowing the antibody to act upon an intermediary (e.g., antigen) that in turn acts upon the cell.

The terms “assaying” and “measuring” and grammatical variations thereof are used interchangeably herein and refer to either qualitative or quantitative determinations, or both qualitative and quantitative determinations. When the terms are used in reference to binding, any means of assessing the relative amount, affinity or specificity of binding is contemplated, including the various methods set forth herein and known in the art. For example, antibody binding can be assayed or measured by an ELISA assay.

Unless otherwise defined, all technical and scientific terms used herein have the same meaning as commonly understood by one of ordinary skill in the art to which this invention relates. Although methods and materials similar or equivalent to those described herein can be used in the practice or testing of the invention, suitable methods and materials are described herein.

All publications, patents, Genbank accession numbers and other references cited herein are incorporated by reference in their entirety. In case of conflict, the present specification, including definitions, will control.

As used herein, singular forms “a”, “and,” and “the” include plural referents unless the context clearly indicates otherwise. Thus, for example, reference to an “antibody” includes a plurality of antibodies and reference to “a treatment or therapy” can include multiple simultaneous, consecutive or sequential treatments or therapies, and so forth.

As used herein, all numerical values or numerical ranges include whole integers within or encompassing such ranges and fractions of the values or the integers within or encompassing ranges unless the context clearly indicates otherwise. Thus, for example, reference to a range of 90-100%, includes any numerical value or range within or encompassing such values, such as 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and any numerical range within such a range, such as 90-92%, 90-95%, 95-98%, 96-98%, 99-100%, etc. In an additional example, reference to a range of 1-5,000 fold includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and any numerical range within such a range, such as 1-2, 5-10, 10-50, 50-100, 100-500, 100-1000, 500-1000, 1000-2000, 1000-5000, etc. In a further example, reference to a range of KD 10⁻⁵ M to about KD 10⁻¹³ M includes any numerical value or range within or encompassing such values.

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EXAMPLES

### Example 1

This example describes various exemplary materials and methods and data.

### Immunohistochemistry (IHC) Analysis

Immunohistochemistry with human IgG on human tissue naturally results in high background staining. Accordingly, BARB3 was labeled with biotin to reduce background.

BARB3 antibody and an unrelated commercial IgG control (Chrompure IgG, Dianova) where labelled with “EZ-Link Maleimide-PEO Solid Phase Biotinylation Kit” from Pierce (Cat-# 21930) and Immunohistochemistry was initiated on different tumor tissue. Parrafin embedded tissue was first deparaffinized by treatment with Xylene 1 for 5 min, Xylene 2 for 5 min, followed by 100% Ethanol 1 for 5 min, 100% Ethanol 2 for 5 min, 70 ml Methanol+500 μl H2O2 for 7 min, 90% Ethanol 1 for 3 min, 90% Ethanol 2 for 3 min, 80% Ethanol 1 for 3 min, 80% Ethanol 2 for 3 min, 70% Ethanol 1 for 3 min, 70% Ethanol 2 for 3 min, and then washed three times with distilled H2O. (1 and 2 mean that the tissues were treated 2 times with the same alcohol, one after the another, in two different tanks.) Tissue was then heated in Citric acid, pH 5,5 in a water-bath at 99.9° C. for 20 min, placed in Tris/NaCl (Tris, 0.6 g/l and NaCl 8.1 g/l, pH 7.4) for 5 min, block with 0.5% BSA in PBS for 60 min, and then washed three times with Tris/NaCl (Tris, 0.6 g/l and NaCl 8.1 μl, pH 7.4). Primary antibody (150 μl per microscope slide) was added, or a negative control biotinylated isotype matched control human antibody (Chrompure IgG, Dianova, Germany) or a positive controlanti-cytokeratin antibody, and incubated for 30 min in a humidified chamber at 37° C. Tissue was washed three times with Tris/NaCl (Tris, 0.6 g/l and NaCl 8.1 g/l, pH 7.4). Secondary antibody: (150 μl per microscope slide) was subsequently added and incubated for 30 min in a humidified chamber at room temperature (for biotinylated antibodies: NeurtrAvidin 1:100 in PBS), washed three times with Tris/NaCl (Tris, 0.6 g/1 and NaCl 8.1 g/l, pH7.4), placed in PBS for 10 min. Tiusse was subsequently incubated with diaminobenzidine (0.05%)-hydrogen peroxide (0.02%) (150 μl per microscope slide) (Sigma, Taufkirchen, München, Germany) for 10 min, washed three times with H2O, washed once with distilled H2O, incubated with hematoxylin/eosin for 5 min, and placed under running tap water 10-15 min. Tissue was then washed again with distilled H2O, and the slides covered with Aquatex (Merck Bioscience). The data is illustrated in Table 1 below:

TABLE 1
IHC Data

| | | | Barb 3 |
|---|---|---|---|
| | Tissue | Carcinoma Type | +/− |
| Malignant | Stomach | Adeno (intestinal) | 2/0 |
| Tissues | | Adeno (Cardio, intestinal) | 1/0 |
| | Lung | Carcinoid (neuron-endocrine) | 1/0 |
| | | Squamous cell | 2/0 |
| | | Lymph Node Metastasis | 1/0 |
| | Colon | Adeno | 1/1 |
| | | Liver Metastasis | 1/0 |
| | Pancreas | Adeno | 3/1 |
| | | Lymph Node Metastasis | 1/0 |
| | Esophagus | Adeno (Barrett Carcinoma) | 1/0 |
| | | Squamous cell | 1/0 |
| | Prostrate | Adeno | 2/0 |
| | Breast | Invasive ductal | 1/0 |
| | | Lung Metastasis | 1/0 |
| | | Lymph Node Metastasis | 1/0 |
| | Melanoma | Metastases of malignant melanoma | 5/0 |
| | | | Barb 3 |
| | Tissue | Cell Type | +/− |
| Healthy | Lung | glandular, alveolar | 0/3 |
| Tissues | Breast | glandular | 0/2 |
| | Colon | glandular | 0/3 |
| | Stomach | glandular | 0/3 |
| | Pancreas | glandular | 0/2 |
| | Esophagus | epithelial | 0/1 |

### FACs Analysis

To analyze tumor cell binding, pancreas cancer cells (BXPC-3), stomach cancer cells (23132/87), and malignant melanoma cells (HTB-69 and CRL-1424) were grown to subconfluency in complete medium, detached with Trypsin/EDTA and incubated on ice for 1 h for recreation. Tumor cells were subsequently incubated on ice with 100 μg/ml BARB3 antibody (diluted in PBS containing 0.01% sodium azide), or 100 μg/ml human isotype-matched control antibody (Chrompure human IgG, Dianova, Hamburg, Germany) for 20 min on ice. Following incubation, cells were incubated with a FITC-labeled rabbit anti-human IgG antibody (1:50, Dako, Germany) for 20 min on ice, and analyzed by flow cytometry (FACScan; Becton Dickinson, USA). The data indicates that BARB3 antibody binds to pancreas (BXPC-3) cancer cells, stomach cancer cells (23132/87) and malignant melanoma cells (HTB-69 and CRL-1424).

### MTT Cell Proliferation Assay

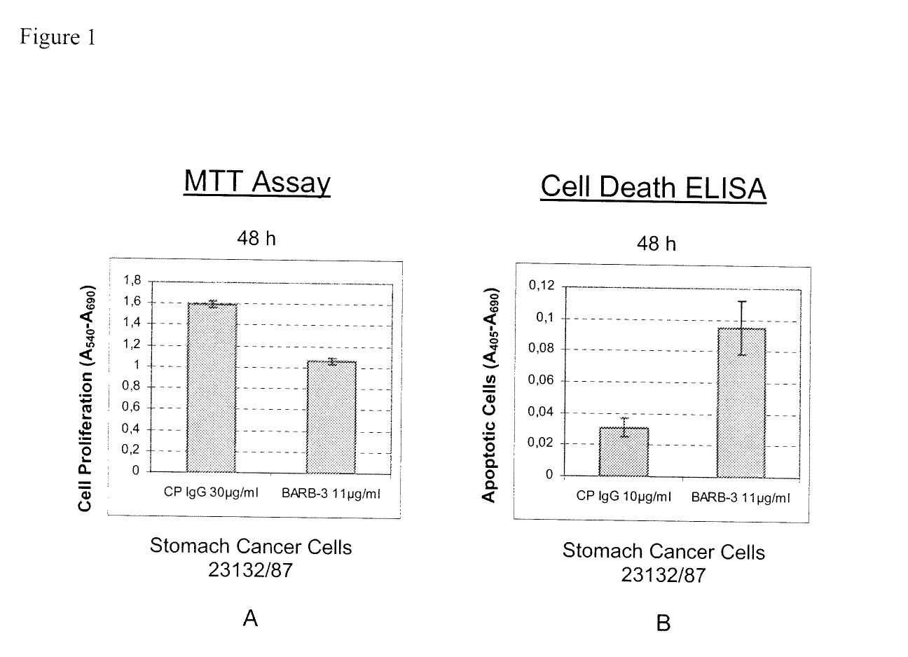

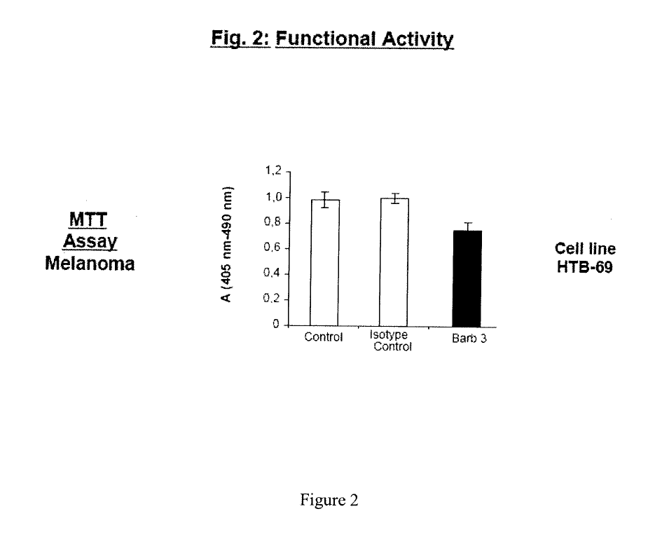

To analyze tumor cell proliferation, stomach cancer cells (23132/87) or malignant melanoma cells (HTB-69) were trypsinized and resuspended in 10 ml of RPMI-1460 medium that contained 10% Fetal Calf Serum (FCS), 1% glutamine, and 1% penicillin/streptomycin (complete medium). Cells (1×10⁴) were plated into 96well plates (24 h) and incubated with purified antibody or supernatants containing antibody for 48 h at 37° C. After incubation, 50 μl MTT (3-(4,5-dimethylthiazol-2-yl)-2,5-diphenyltetrazolium bromide) solution (5 mg/ml in PBS), (Sigma, Taufkirchen, München, Germany) was added to each well. The 96-well plate was incubated for 30 minutes at 37° C. and centrifuged for 10 minutes at 2800 rpm, the supernatant aspirated, 150 μl of DMSO added to each well, and the cell pellet was resuspended. Absorption was determined at a wavelength of 540 nm and at a reference wavelength of 690 nm in an ELISA reader. The data is illustrated in FIGS. 1A and 2.

### Cell Death (Apoptosis) Assay

Cell Death Detection Elisaplus (Roche, Mannheim, Germany) was used to analyze the extent to which the antibodies induce apoptosis. This assay is based upon a quantitative sandwich-enzyme-immunoassay principle using mouse monoclonal antibodies directed against DNA and histones, respectively. This assay allows the specific determination of mono- and oligo-nucleosomes which are released into the cytoplasm of cells which die from apoptosis. In brief, cells (1.0×10⁴) were plated in 96 well plates and incubated with antibody at 37° C. and 7% CO2 for 24/48 hours. After incubation, the cells were centrifuged at 200 g for 10 minutes, the supernatant was aspirated and 200 μl of lysis buffer added, which resulted in the lysis of cells following a 30 minute incubation at room temperature. After centrifuging again, 20 μl of the supernatant was added to streptavidin-coated micro-titer plates and 80 μl of the immuno-reagent (1/20 Anti-DNA-peroxidase (anti-DNA-POD) antibody, which reacts with the DNA components of the nucleosomes, 1/20 Anti-Histone Biotin, 18/20 incubation buffer) was added. A positive control and the blank included in the manufacturers test kit were used. After plates were incubated for 2 hours while being mixed at approximately 250 rpm, each well was washed three times with 250 μl of incubation buffer, followed by addition of 100 μl of ABTS solution (1 ABTS (2,2′-Azino-di[3-ethyl-benz-thiazolin-sulfonat) tablet in 5 ml substrate buffer) to each well. The plates were mixed again and intensity of antibody-induced apoptosis is reflected in the intensely green precipitate. The color intensity was determined using an ELISA reader at a wavelength of 415 nm against a reference wavelength of 490 nm. Based on this color intensity, the intensity of the antibody-induced apoptosis was calculated. The data is illustrated in FIG. 1B.

### Immunofluorescence

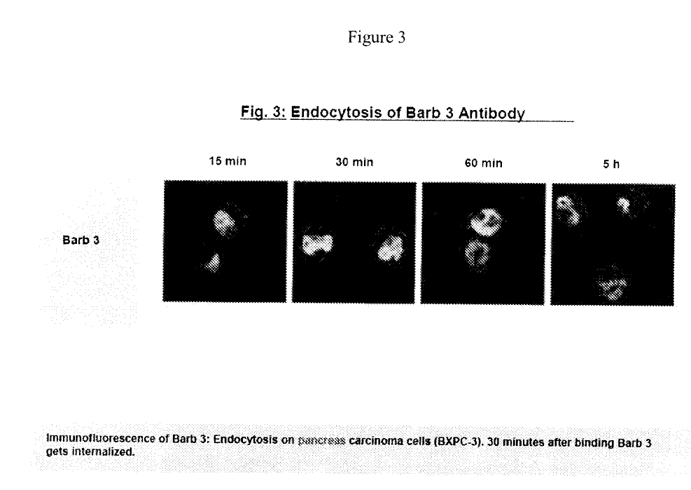

Endocytosis was determined for BARB3 on human pancreas carcinoma cell-line BXPC-3. BARB3 antibody (purified) was conjugated with Fluorescent orange 548 reactive (Fluka, Buchs, Switzerland). Conjugated BARB3 antibody at a final concentration of 40 μg/ml was directly given to 1×10⁶ cells and incubated for indicated times at 37° C. Cells were harvested, rinsed and resuspended in phosphate buffer saline pH 7,4 (PBS). 100 μl of each cell suspension was fixed on slides. Finally the slides were mounted with Fluorescent Mounting Medium (DakoCytomation, Carpinteria, USA) and analyzed by confocal microscopy. The data is illustrated in FIG. 3.

### Example 2

This example describes data that may indicate that N-linked sugar residues could be involved in BARB3 binding to antigen.

### Glycosidase Assay on Cytospins

To determine whether O- or N-linked sugar residues are potentially involved in BARB3 binding to tumor cells, cytospin preparations of BXPC-3 cancer cells were incubated with N- or O-glycosidase. In brief, 4×10⁵ human pancreas carcinoma cells (BXPC-3) were resuspended in 1 ml Dulbecco's phosphate buffered saline pH 7.2 (Sigma, Taufkirchen, Germany) and incubated with 10 U/ml N-glycosidase or 40 mU/ml O-glycosidase (both Roche Applied Science, Mannheim, Germany) for 2 hours at 37° C. Untreated cells in Dulbecco's phosphate buffered saline served as control. Cytospins were prepared and immunohistochemical staining with biotinylated BARB3 antibody (100 μg/ml) was performed.

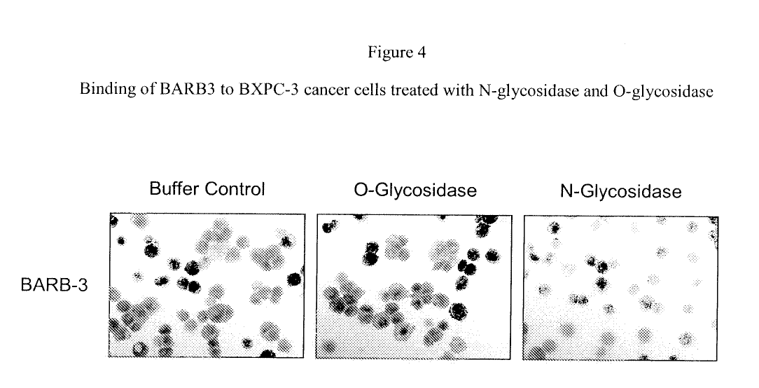

After treatment of the cells, binding of BARB3 was evaluated by immunohistochemical staining (FIG. 4). The data show a clear reduction of surface binding of BARB3 on cells treated with N-glycosidase, while treatment with O-glycosidase has no effect on BARB3 binding. BARB3 could therefore bind to an eptiope that includes or consists of a carbohydrate moiety that is removed or modified by O-glycosidase treatment.

### Example 3

This example describes data indicating that prohibitin is a putative target antigen to which BARB3 binds.

CRL1424 cell membrane extracts were prepared and immunoprecipitated (IP) with BARB3 antibody. In brief, medium was removed from cultured cells and the cells were washed three times with pre-cooled PBS. 5 mL pre-cooled PBS was added to a 15 cm culture dish and cells were scraped using a cell scraper. The cell suspension was transferred to a 50 mL tube and centrifuged at 1,300 rpm for 5 minutes. Cell pellets from different culture dishes were collected and again washed with PBS. After centrifugation with 1,300 rpm for 5 minutes cells were resuspended in hypoton buffer (10 mL/1 g cell pellet). The cell suspension was incubated for 30 minutes on ice with vortexing every 5 minutes and then freeze/thawed on liquid nitrogen 5 times. To sediment the cell debris the suspension was centrifuged for 10 minutes at 13,000 rpm at 4° C. The resulting supernatant was then centrifuged in an ultracentrifuge for 45 minutes at 125,000×g at 4° C. The resulting pellet of 4 g of suspension was resuspended in 1 mL lysis buffer and solved with a short pulse (2 sec) sonification. The sonicated suspension was then centrifuged for 10 minutes at 13,000 rpm at 4° C., and the supernatant containing the membrane fraction was transferred to a fresh 1.5 mL tube.

Immunopurification of the cell membrane extracts was subsequently performed with μ Columns and μMACS Separator (Miltenyi Biotec). In brief, to 150-300 μL membrane preparation or 300-400 μL full lysate, 1.5 μL of monoclonal human BARB3 antibody and 50 μL of Protein G Micro Beads (magnetic labeled) were added and was filled with lysis buffer to a total volume of 800 μL. For immunoprecipitation with human IgGs, 100 μL of anti human IgG Micro Beads and 50 μg of purified human IgG were added to the lysate. The suspension was incubated for 30 minutes rotating with 16 rpm at 4° C. Miltenyi μColumns were placed in the magnetic field of the μMACS Separator. Columns were prepared by rinsing with 200 μL of lysis buffer. Cell lysate was applied onto the column. After the lysate ran through the columns were washed with 5×200 μL lysis buffer. For elution 20 μL of pre-heated (95° C.) 1×SDS gel loading buffer (50 mM Tris HCl, pH 6.8; 50 mM DTT; 1% SDS; 0.005% bromphenol blue; 10% glycerol) was applied onto the column and incubated for 5 minutes at room temperature. A fresh collection tube was placed under the column and the column was eluted with another 50 μL of pre-heated (95° C.) 1×SDS gel loading buffer.

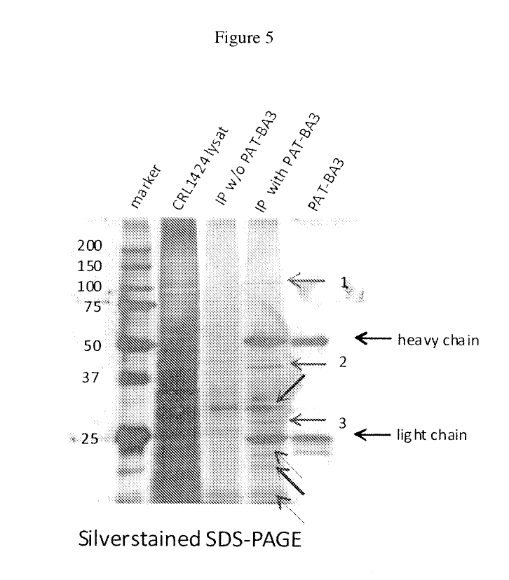

The immunoprecipitated antigen(s) were fractionated via SDS-PAGE and compared to various controls, immunoprecipitation without BARB3 antibody, BARB3 antibody (FIG. 5, right lane, heavy and light chain indicated), and CRL 1424 crude lysate. The SDS-PAGE was subsequently silver stained (FIG. 5). Gel slices containing the region indicated by horizontal arrows 1, 2 and 3 in the BARB3 immunoprecipitated lane were excised, protein eluted from the gel, subjected to Matrix Assisted Laser Desorption/Ionization Time-of-Flight (MALDI-TOF) mass spectrometry sequence analysis (ProteoMetrics, LLC). Sequence analysis revealed that Arrows 1 and 2 were BARB3 IgG fragments. A comparison of the sequence information obtained from the gel slice indicated by Arrow 3 to the protein database revealed human prohibitin.

To validate prohibitin as a potential target, CRL1424 cell membrane extracts were prepared and immunoprecipitated with anti-Prohibitin rabbit polyclonal antibody (Abeam, order-number ab28172) and subjected to SDS-PAGE fractionation as described. The fractionated proteins were transferred to a membrane and analyzed by Western blotting using a BARB3 antibody. The blots did not reveal an increased signal for prohibitin, which may have been due to loss of BARB3 antigenic site from denaturating conditions.

To ascertain expression of prohibitin on the surface of cancer cells, HTB 69 (melanoma) and BxPC-3 (pancreatic) cells were incubated with 100 ug/ml anti-prohibitin or BARB3 antibody and analyzed for binding via FACS analysis. For FACS, the following protocol was used: Cells transfection were dissolved with cell dissociation solution for 10 minutes, resuspended in complete growth medium, centrifuged at 800 g for 5 minutes. The cell pellet was resuspended in fresh complete medium, counted, and adjusted to 2×10⁵/ml in complete medium. Cells were incubated on ice for 30 min, and 1 ml cells per Eppendorf-tube dispensed. Cells were washed with PBS, centrifuged at 800 g (4° C.) and resuspended in 500 μl PBS. To cells, 100 μg/ml first antibody, Anti-Prohibitin murine IgG (Medac, clone number: II-14-10, order number: MS-261-P1), or BARB-3, was added. Negative controls were Isotype control and without 1^{st} antibody. After incubation, cells were washed once with PBS, and then secondary antibody (anti-human IgG-FITC, or anti-mouse IgG FITC diluted 1:50 in 200 μl) added, and incubated for 30 min in the dark. Cells were subsequently washed twice with PBS and transferred to a FACS tube in 250 μl PBS. Antibody binding was measured with a FACS Scan (Beckmann-Coulter), and data analysed with the free software WinMDI 2.8.

The data revealed that anti-prohibitin binds to cell surface of HTB 69 cells. Thus, analogous to the target that BARB3 binds, prohibitin is also expressed on the surface of cancer cells.

### Example 4

This example describes studies to evaluate the half life of BARB3 in nude mice.

In brief, a single nude mouse for each time point was injected IP with 20 ug/kg (0.4 mg/mouse) BARB3. In one short terms study, serum was withdrawn from each mouse at 1, 3, 6, 24, 48 and 72 hours. In a longer term study, serum was withdrawn from each mouse at 1, 6, 24, 72, 120 and 168 hours. The amount of BARB3 antibody was determined by ELISA, and serum concentrations (ug/ml) are shown below.

| Time | Concentration (ug/ml) | stddev |
|---|---|---|
| 1 | 89.65 | 27.42 |
| 3 | 100.7 | 35.68 |
| 6 | 70.96 | 9.79 |
| 24 | 83.79 | 19.12 |
| 48 | 113.53 | 6.57 |
| 72 | 99.93 | 8.22 |
| 1 | 46.96 | 50.20 |
| 6 | 171.54 | 77.58 |
| 24 | 161.35 | 13.71 |
| 72 | 110.16 | 42.89 |
| 120 | 68.94 | 5.55 |
| 168 | 75.02 | 13.94 |

1.-5. (canceled)

6. An isolated or purified antibody or functional fragment thereof that binds to a cell or to an antigen that intact BARB3 antibody, as represented by DSMZ Deposit No. DSM ACC2859, or heavy and light chain sequences set forth as SEQ ID NOs:1 and 2, binds.

7. The isolated or purified antibody or functional fragment of claim 6, wherein the antibody or functional fragment binds to an adenocarcinoma cell or a squamous cell carcinoma to which BARB3 antibody, as represented by DSMZ Deposit No. DSM ACC2859, or heavy and light chain sequences set forth as SEQ ID NOs:1 and 2, binds.

8. The isolated or purified antibody or functional fragment of claim 6, wherein the antibody or functional fragment binds to one or more of a stomach adenocarcinoma cell, a lung adenocarcinoma cell, a pancreas adenocarcinoma cell, a colon adenocarcinoma cell, a breast adenocarcinoma cell, an esophagus squamous cell carcinoma, to which BARB3 antibody, as represented by DSMZ Deposit No. DSM ACC2859, or heavy and light chain sequences set forth as SEQ ID NOs:1 and 2, binds.

9. The isolated or purified antibody or functional fragment of claim 6, wherein the antibody or functional fragment binds to a human adenocarcinoma, squamous cell carcinoma, carcinoid carcinoma, ivasive ductal carcinoma, germ cell carcinoma of stomach, lung, colon, pancreas, esophagus, prostate, breast or testis to which BARB3 antibody, as represented by DSMZ Deposit No. DSM ACC2859, or heavy and light chain sequences set forth as SEQ ID NOs:1 and 2, binds.

10. The isolated or purified antibody or functional fragment of claim 6, wherein the antibody or functional fragment binds to a pancreas cancer cell line BXPC-3 (ATCC Deposit No. CRL-1687) or a stomach cancer cell line 23132/87 (DSMZ Deposit No. ACC 201) that intact BARB3 antibody binds.

11. An isolated or purified antibody or a functional fragment thereof comprising a heavy or a light chain variable region sequence with about 60% or more identity to a heavy or light chain sequence variable regions as set forth in SEQ ID NOs:1 and 2.

12.-15. (canceled)

16. The antibody or function fragment of claim 6, wherein the antibody or functional fragment inhibits or reduces proliferation, or stimulates or induces apoptosis, of one or more of a stomach adenocarcinoma cell, a lung adenocarcinoma cell, a pancreas adenocarcinoma cell, a colon adenocarcinoma cell, a breast adenocarcinoma cell, or an esophagus squamous cell carcinoma, a human adenocarcinoma, human squamous cell carcinoma, human carcinoid carcinoma, human invasive ductal carcinoma, or a human germ cell carcinoma in any of stomach, lung, colon, pancreas, esophagus, prostate, breast or testis, or a pancreas cancer cell line BXPC-3 (ATCC Deposit No. CRL-1687) or a stomach cancer cell line 23132/87 (DSMZ Deposit No. ACC 201).

17. The antibody or functional fragment of claim 6, wherein the antibody is polyclonal or monoclonal.

18. The antibody or functional fragment of claim 6, wherein the antibody is selected from IgG, IgA, IgM, IgE and IgD.

19. (canceled)

20. The antibody or functional fragment of claim 6, wherein the antibody or functional fragment has a binding affinity within about 1-5000 fold of the binding affinity of BARB3 antibody, as represented by DSMZ Deposit No. DSM ACC2859, or heavy and light chain sequences set forth as SEQ ID NOs:1 and 2, for binding to a neoplastic, cancer, tumor or metastatic cell.

21.-23. (canceled)

24. The antibody or functional fragment of claim 6, wherein the antibody or functional fragment has a binding affinity within about KD 10⁻⁵ M to about KD 10⁻¹³ M for binding to a neoplastic, cancer, tumor or metastatic cell.

25.-27. (canceled)

28. The antibody or functional fragment of claim 6, wherein the functional fragment is selected from Fab, Fab′, F(ab′)₂, Fv, Fd, single-chain Fv (scFv), disulfide-linked Fvs (sdFv), V_{L} and V_{H} domain fragments, trispecific (Fab₃), bispecific (Fab₂), diabody ((V_{L}-V_{H})₂ or (V_{H}-V_{L})₂), triabody (trivalent), tetrabody (tetravalent), minibody ((scFv-CH₃)₂), bispecific single-chain Fv (Bis-scFv), IgGdeltaCH2, scFv-Fc and (scFv)₂-Fc.

29.-31. (canceled)

32. The antibody or functional fragment of claim 6, wherein the cell or cell line expresses a cell membrane bound prohibitin isoform, or the antigen comprises a cell membrane bound prohibitin isoform.

33. The antibody or functional fragment of claims 11, wherein the antibody or functional fragment competes with BARB3 antibody, as represented by DSMZ Deposit No. DSM ACC2859, or heavy and light chain sequences set forth as SEQ ID NOs:1 and 2, for binding to prohibitin.

34. The antibody or functional fragment of claim 33, wherein the prohibitin comprises a cell membrane bound prohibitin isoform.

35. A heavy chain sequence comprising SEQ ID NO:1 or SEQ ID NO:2.

36. A host cell that expresses the antibody or functional fragment of claim 6, or the heavy or light chain sequence of SEQ ID NO:1 or 2.

37.-58. (canceled)

59. A pharmaceutical composition comprising the antibody or functional fragment of claim 6, or the heavy or light chain sequence of SEQ ID NO:1 or 2, and a pharmaceutically acceptable carrier or excipient.

60-108. (canceled)

109. An isolated or purified antigen, wherein the antigen comprises an amino acid sequence at least 65%, 70%, 75%, 80%, 85%, 90%, 95%, or more identical to prohibitin protein sequence, wherein the prohibitin is a cell membrane bound isoform.

110. The isolated or purified antigen of claim 109, wherein the antigen is expressed by a tumor or cancer cell.

111. The isolated or purified antigen of claim 110, wherein the tumor or cancer cell is a pancreas cancer or tumor cell, a colon cancer or tumor cell, or a stomach cancer or tumor cell.

112. The isolated or purified antigen of claim 110, wherein the tumor or cancer cell comprises BXPC-3 cells, or HTB-69 cells.

113. The isolated or purified antigen of claim 109, wherein a BARB3 antibody, as represented by antibody produced by hybridoma DSMZ Deposit No. DSM ACC2859, or heavy and light chain sequences set forth as SEQ ID NOs:1 and 2, binds to the antigen.

114. The isolated or purified antigen of claim 109, wherein the antigen comprises a cell membrane bound prohibitin isoform.

115. The isolated or purified antigen of claim 109, wherein the antigen includes a carbohydrate moiety.

116.-119. (canceled)

120. An isolated or purified antibody or subsequence thereof that competes for binding of BARB3 antibody, as represented by antibody produced by hybridoma DSMZ Deposit No. DSM ACC2859, or heavy and light chain sequences set forth as SEQ ID NOs:1 and 2, for binding to the antigen of claim 109.

121. The antibody or subsequence thereof of claim 120, wherein binding of the antibody or subsequence thereof to the antigen after treatment with an N-glycosidase is reduced as compared to binding of the antibody or subsequence thereof to the untreated antigen.

122. The antibody or subsequence thereof of claim 120, wherein binding of the antibody or subsequence thereof to the antigen or expressed on cells after transfection with an antisense nucleic acid of prohibitin is reduced as compared to binding of the antibody or subsequence thereof to the antigen expressed on cells not transfected with the antisense nucleic acid.

123. The antibody or subsequence thereof of claim 120, wherein the antibody or subsequence thereof is a mammalian antibody.

124. The antibody or subsequence thereof of claim 120, wherein the antibody or subsequence thereof is a human or humanized antibody.

125. The antibody or subsequence thereof of claim 120, wherein the antigen comprises a cell membrane bound prohibitin isoform.

126. The antibody or subsequence thereof of claim 120, wherein the antibody has a binding affinity for prohibitin expressed by a tumor or cancer cell greater than the binding affinity for prohibitin expressed in a non-tumor or non-cancer cell.

127. The antibody or subsequence thereof of claim 120, wherein the antibody has a binding affinity for a cell membrane bound prohibitin isoform greater than the binding affinity for prohibitin expressed in a non-tumor or non-cancer cell.

128.-129. (canceled)
